(19) United States
(12) Patent Application Publication
(10) Pub. No.: US 2004/0215010 A1
(43) Pub. Date: Oct. 28, 2004

(54) Universal solid supports for solid phase oligosynthesis and methods for their preparation and use

(76) Inventor: Viktor Kumarev, Villemonble (FR)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/297,681

(22) PCT Filed: Jun. 12, 2001
(86) PCT No.: PCT/IB01/01392

(30) Foreign Application Priority Data
Jun. 13, 2000 (US) 60211344

(51) Int. Cl. C07H 21/04; C08G 63/91; C08G 63/48

(52) U.S. Cl. 536/25.3; 525/54.2

(57) ABSTRACT

This invention pertains to solid-phase oligosynthesis, and more particularly to universal solid supports for solid-phase oligonucleotide synthesis, methods for their preparation, and methods for their use. One aspect of the invention pertains to a method for the preparation of a universal solid support suitable for use in solid-phase oligosynthesis, which method comprises the steps of: (a) reacting a pendant functional group (e.g., —NH2) of a solid support (e.g., CPG) with a linker reagent (e g., oxalyl chloride), thereby forming a pendant linker group (e.g., —C(═O)Cl); (b) removing at least a portion of excess unreacted linker reagent by evaporation (e.g., using a rotavapor apparatus); and, (c) reacting said pendant linker group with a cyclic reagent (e.g., protected inosine), thereby forming a pendant cyclic group.

### TECHNICAL FIELD

This invention pertains generally to the field of solid-phase oligosynthesis, and more particularly to universal solid supports for solid-phase oligonucleotide synthesis, methods for their preparation, and methods for their use.

### BACKGROUND

The widespread use of modified and unmodified oligonucleotides as probes and primers in the field of molecular biology as well as the potential applications of antisense and antigene oligonucleotides as therapeutics make the continuous development of new and improved synthetic methods for these molecules increasingly important.

Some procedures in oligonucleotide synthesis require that the solid support contain the first nucleoside which is destined to become the nucleoside at the 3′-terminus of the synthetic oligonucleotide. Such procedures require an inventory of all eight regular nucleoside supports (four for DNA and four for RNA) to be maintained. Moreover, unusual nucleosides are often available only as phosphoramidites and not as supports, and thus oligonucleotides with unusual nucleosides at the 3′-terminus can not be readily prepared. Furthermore, another drawback of such methods is the potential for an error in the selection of the column containing the 3′-nucleoside. Although this potential for error may be fairly low in regular column-type synthesizers, it is especially significant in the new generation of parallel synthesizers where 24, 96, or 192 wells may contain the four supports in a defined grid.

In order to alleviate these problems, various “universal” solid supports have been developed. These universal solid supports can be used to synthesize any oligonucleotide regardless of the nature of the 3′-terminal nucleoside.

DeBear et al., 1987, describe a universal solid support in which a protected uridine group is linked via a succinyl group to a solid support. The support is prepared by, first, reacting protected uridine with succinic anhydride in the presence of 4-dimethylaminopyridine, second, activating the resulting succinate half-esters with p-toluenesulfonyl nitrotriazole, and third, reacting the active esters with pendant amino groups of a solid support. These steps require more than two days, and include purification by washing with pyridine, methanol, and chloroform, followed by vacuum desiccation. In no instance was excess reagent removed by evaporation or solely by evaporation.

Webb et al., 1987, describe methods of preparing universal solid supports which employ solid supports bearing oxirane, aziridine, or episulfide groups, linker compounds having nucleophilic groups, for example, free amino groups, and activated nucleosides bearing pentachlorophenyl succinates. Although the universal solid supports are apparently prepared without the need for chromatographic separation (col. 4, lines 41-42), the synthesis does require many purification steps (e.g., filtering, washing, drying) and at least two days time (Example 1). Nowhere is there any mention of using evaporative methods for removal.

Nelson et al., 1989, describe an adapter compound which has two different protected groups, one (—O-DMT) for oligonucleotide synthesis and one (—NHFmoc) for other use, e.g., label attachment. The adapter is first derivatized to bear an activated succinyl group, and subsequently coupled to the solid support. These steps require many purification steps (e.g., washing, drying, filtering). In no instance was excess reagent removed by evaporation or solely by evaporation. Succinylation and coupling to the support required more than 20 hours, and often much longer.

Letsinger et al., 1992, describe universal solid supports employing, for example, oxalyl linker arms (see FIG. 1C and FIG. 2 therein). The oxalyl linker arm is introduced using an activated oxalyl derivative (col. 4, lines 62-64). Oxalyl chloride is converted to a less reactive triazolide derivative prior to use (col. 5, lines 30-37). The nucleoside loaded oxalyl support apparently can be prepared in one to eight hours (col. 5, lines 43-45), and required 2.5 hours in Example 1 (col. 6, lines 44-56). The universal solid support was prepared in a syringe apparatus, with reactants (either liquid, or in solution) being injected, excess reactants subsequently ejected, and the residue rinsed or washed several times with solvent (e.g., acetonitrile, pyridine, methylene chloride, diethyl ether). Many purification steps (e.g., rinsing, washing) are required. Nowhere is there any mention of using evaporative methods for removal.

Arnold, 1994 and Arnold, 1996, describe universal solid supports which rely on selective oxidation in order to achieve final cleavage of the oligonucleotide. In each case, the primer moiety (e.g., riboside), which links the oligonucleotide to the solid support, possesses one or more oxidizable substituents (col. 6, lines 52-62). The synthesis requires many purification steps (e.g., washing, filtering, drying, centrifugation, partitioning, and column chromatography) (see Examples 1, 3, 5, and 7). In no instance was excess reagent removed by evaporation or solely by evaporation. Forming the linkages between the solid support, the linker group, and the primer group required more than 6 hours, and often much longer.

Vu et al., 1995, describe a number of different primers which have two different protected groups, one for oligonucleotide synthesis and one for other use, e.g., label attachment. In each case, the primer is first attached to a linker group (succinylation) and subsequently coupled to the support in the final step, and these steps required many purification steps (e.g., filtering, washing, drying, and column chromatography) (see synthesis for compounds 4, 5, & 6; 12 & 13; 18 & 19; 25 & 26; and 31 & 32). In no instance was excess reagent removed by evaporation or solely by evaporation. Succinylation and coupling to the support required more than 6 hours, and often much longer.

Schwartz et al., 1995, describe a universal adapter compound which can convert a nucleoside-bearing solid support to a universal solid support. The linker compound, a fully protected 2′(3′)-O-dimethoxytrityl-3′(2′)-O-benzoyluridine-5′-O-(2-cyanoethyl-N,N-diisopropylphosphoramidite), is linked to the nucleoside-bearing solid support using conventional phosphoramidite methods.

McLean et al., 1997, describe universal solid supports in which a homocyclic (e.g., cyclohexyl) or heterocyclic (e.g., furan, pyran) ring bearing a nucleophilic group (e.g., hydroxyl or protected hydroxyl) is tethered to a solid support by a spacer group (e.g., succinyl). The universal solid supports are synthesized by first attaching a linker group to the cyclic group, and then attaching the linker-cyclic group to the solid support. The synthesis requires many purification steps (e.g., filtering, washing, drying, column chromatography, partitioning) (see Examples 1&2, 13&14, 16&17, 19, 20&21, 23&24, and 26). In no instance was excess reagent removed by evaporation or solely by evaporation. Coupling of the linker group to the cyclic group, and subsequent coupling to the solid support required more than 36 hours, and often much longer.

Lyttle et al., 1996, describe universal solid supports in which a propyl group bearing a protected hydroxy group (as —O-DMT) for oligosynthesis, and a protected amino group (as allyl carbamate) to assist with the cleavage mechanism, wherein the propyl group is bound to the solid support via a succinyl group. The supports are prepared using a universal linker, 1-O-(4,4′-dimethoxytrityl)-2-O-succinoyl-3-N-allyloxycarbonylpropane. Formation of the succinate derivative (compound 5 to compound 1) required over 12 hours and several purification steps, including column chromatography. In no instance was excess reagent removed by evaporation or solely by evaporation. Subsequent coupling to a solid support required another 12 hours.

Scheuer-Larsen et al., 1997, describe a universal solid support in which a D-ribose group, bearing a protected hydroxy group (as —O-DMT) for oligonucleotide synthesis and a protected hydroxyl group (as chloroacetyl), is linked to a solid support by a succinyl group. Formation of the succinate derivative and subsequent coupling the solid support (compound 5 to 6, steps f and g, in Scheme 1) required several purification steps (e.g., washing, partitioning, and drying) and more than 44 hours. In no instance was excess reagent removed by evaporation or solely by evaporation.

Reddy et al., 1997, describe universal solid supports in which a ring moiety (—R³, preferably a riboside), bearing vicinal groups (—XR¹ and —YR²), is linked, for example, by an ester linkage, a phosphate linkage (e.g., Example 8), a phosphoramidate linkage (e.g., Example 11), or an ester linkage (e.g., Example 13), to a solid support, typically via a succinyl group. In Example 13, the solid support is derivatized with succinic anhydride and subsequently reacted with 2′,3′-protected uridine. The process requires more than three days, and many purification steps (e.g., filtering, washing, and drying). In no instance was excess reagent removed by evaporation or solely by evaporation.

Known methods for the preparation and use of the universal solid supports, including those described above, are complex and time-consuming, and there is a great need for universal solid supports for oligonucleotide synthesis which may be prepared easily and quickly, in fewer steps, with fewer and simpler purification steps, and which facilitate simple and fast cleavage and deprotection of the synthesized oligonucleotide.

By employing a method in which (a) the solid support is first reacted with a linker reagent (to form a pendant linker group) and then reacted with a cyclic reagent (to form a pendant cyclic group), and (b) the linker reagent is selected both (i) to be volatile, or, more preferably, a volatile liquid, and (ii) to yield a linkage group which is readily cleaved under mild conditions, it is possible to greatly simplify the synthesis by removing, or substantially removing, excess linker reagent by evaporation, even solely by evaporation. Until now, such methods have not been taught or suggested.

Thus, one aim of the present invention is the provision of methods for the preparation of a universal solid support for oligonucleotide synthesis, which methods are rapid (e.g., less than about 3 hr).

Another aim of the present invention is the provision of methods for the preparation of a universal solid support for oligonucleotide synthesis, which methods employ only one purification step (e.g., filtration), for example, for the removal of excess unreacted cyclic reagent, capping reagent (if applicable), and by-products thereof.

Another aim of the present invention is the provision of methods for the preparation of a universal solid support for oligonucleotide synthesis, which methods give such supports in high yield and with high loading (e.g., about 60 μmole/g).

Another aim of the present invention is the provision of methods for the preparation of a universal solid supports for oligonucleotide synthesis, which methods permit the use of a substantially reduced cleavage time following oligonucleotide synthesis (e.g., about 1 hr at 75 Ec).

Another aim of the present invention is the provision of methods for the preparation of a universal solid supports for oligonucleotide synthesis, which methods satisfies one or more of the above aims.

### BRIEF DESCRIPTION OF THE DRAWINGS

FIG. 1 is a chemical scheme illustrating the preparation of a preferred class of universal solid supports of the present invention.

FIG. 2 is a chemical scheme illustrating the preparation of the universal solid support described in the Example.

FIG. 3 is a chemical scheme illustrating the first cycle of a phosphoramidite oligonucleotide synthesis method, starting with one preferred universal solid support of the present invention.

FIG. 4 is a chemical scheme illustrating the cleavage step for an oligonucleotide grown in a 3′65′ direction using a preferred universal solid support of the present invention.

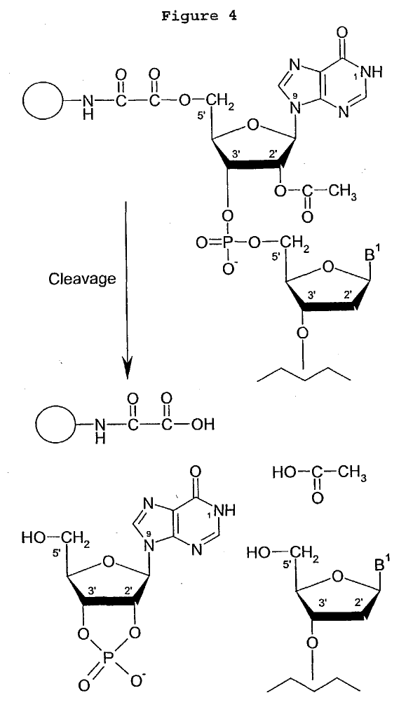

FIG. 5 is a chemical scheme illustrating the cleavage step for an oligonucleotide grown in a 5′63′ direction using a preferred universal solid support of the present invention.

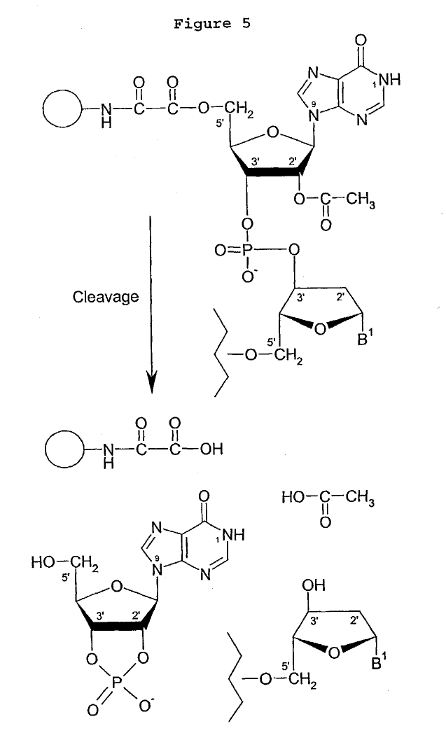

### SUMMARY OF THE INVENTION

One aspect of the present invention pertains to methods for the preparation of a universal solid support for oligosynthesis, which support is useful, for example, in solid-phase oligonucleotide synthesis.

Thus, in one embodiment, the present invention pertains to a method for the preparation of a solid support suitable for use in solid-phase oligosynthesis, which method comprises the steps of:

(a) reacting a pendant functional group of a solid support with a linker reagent, thereby forming a pendant linker group;

(b) removing at least a portion of excess unreacted linker reagent by evaporation; and,

(c) reacting said pendant linker group with a cyclic reagent, thereby forming a pendant cyclic group.

In one embodiment, the present invention pertains to a method for the preparation of a solid support suitable for use in solid-phase oligonucleotide synthesis, which method comprises the steps of:

(a) reacting a pendant functional group of a solid support with a linker reagent, thereby forming a pendant linker group;

(b) removing at least a portion of excess unreacted linker reagent by evaporation;

(c) reacting said pendant linker group with a cyclic reagent, thereby forming a pendant cyclic group; and,

(d) removing at least a portion of excess unreacted cyclic reagent and/or by-products thereof.

In one embodiment, the method further comprises, after said step of reacting a pendant functional group of a solid support with a linker reagent, a step of:

(i) reacting at least a portion of any remaining pendant functional groups of said solid support with a capping reagent, thereby forming pendant capped functional groups.

In one embodiment, the method further comprises, after said step of removing at least a portion of excess unreacted linker reagent by evaporation, a step of:

(i) reacting at least a portion of any remaining pendant functional groups of said solid support with a capping reagent, thereby forming pendant capped functional groups.

In one embodiment, the method further comprises, after said step of reacting said pendant linker group with a cyclic reagent, a step of:

(i) reacting at least a portion of any remaining pendant linker groups of said solid support with a capping reagent, thereby forming-pendant capped linker groups.

In one embodiment, said step of removing at least a portion of excess unreacted linker reagent by evaporation is achieved with the application of reduced pressure and/or increased temperature.

In one embodiment, said step of removing at least a portion of excess unreacted linker reagent by evaporation involves removing a substantial portion of excess unreacted linker reagent.

In one embodiment, said step of removing at least a portion of excess unreacted linker reagent is achieved solely by evaporation.

In one embodiment, one or more steps of said method are performed using an evaporation apparatus. In one embodiment, said method is performed using a rotavapor apparatus or a distillation apparatus. In one embodiment, said method is performed using a rotavapor apparatus.

In one embodiment, said linker reagent has the formula:

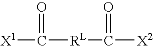

wherein:

X¹ and X² are independently —Cl, —Br, or —I; and,

R^{L} denotes a covalent bond or a divalent group which is an organic group comprising from 1 to 10 carbon atoms and from 0 to 5 heteroatoms selected from N, O, and S;

and wherein said linker reagent is a volatile liquid.

In one embodiment, said linker reagent has the formula:

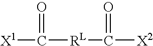

wherein X¹ and X² are independently —Cl, —Br, or —I, and n is an integer from 0 to 2.

In one preferred embodiment, said linker reagent is:

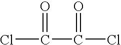

In one embodiment, said pendant functional group of said solid support is -J¹-H, wherein J¹ is —NH—, —O—, or —S—. In one embodiment, said pendant functional group of said solid support is —OH.

In one embodiment, said cyclic reagent has the formula:

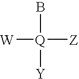

wherein:

Q denotes a cyclic group;

B denotes a base group;

W denotes a reactive conjugating group;

Y denotes an oligosynthesis group; and,

Z, if present, denotes an auxiliary group.

In one embodiment, said cyclic reagent has the formula:

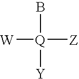

wherein:

Q denotes a cyclic group, which has a single non-aromatic ring, which ring has from 5 to 7 ring atoms, which ring atoms are:

(a) all carbon atoms; or,

(b) carbon atoms and one or two heteroatoms selected from oxygen, nitrogen, and sulfur;

W denotes a reactive conjugating group and is —OH, —NH₂, or —SH;

B denotes a base moiety, which is a purine or pyrimidine or a derivative or analog thereof;

Y denotes an oligosynthesis group, which is —OH, —NH₂, or —SH, or a protected form thereof; and,

Z, if present, denotes an auxiliary group, which is —OH, —NH₂, or —SH, or a protected form thereof;

wherein said W, B, Y, and Z, if present, are each separately attached to a carbon ring atom of said Q, either directly, via a covalent bond, or indirectly, via an intermediate covalent linkage selected from —CH₂— and —CH₂CH₂—.

In one preferred embodiment, said cyclic reagent is:

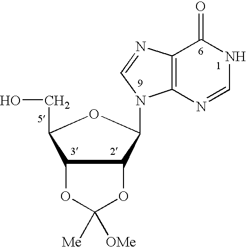

In one embodiment, said step of reacting said pendant linker group with a cyclic reagent is performed in the present of an added base. In one embodiment, said added base is dimethylaminopyridine (DMAP) or N-methylimidazole (NMI).

Another aspect of the present invention pertains to a solid support suitable for use in solid-phase oligosynthesis, preferably oligonucleotide synthesis, which support has the following formula:

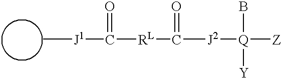

wherein J¹, J², R^{L}, Q, B, Y, and Z are as defined herein.

In one preferred embodiment, the solid support has the following formula:

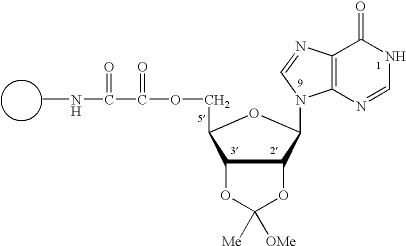

Another aspect of the present invention pertains to a solid support suitable for use in solid-phase oligosynthesis, preferably oligonucleotide synthesis, prepared by a method as described herein.

Another aspect of the present invention pertains to a method of oligosynthesis, preferably oligonucleotide synthesis, which method employs a method for the preparation of a universal solid support as described herein.

Another aspect of the present invention pertains to a method of oligosynthesis, preferably oligonucleotide synthesis, which method employs a universal solid support as described herein.

Another aspect of the present invention pertains to an oligonucleotide which has been prepared using a method of oligosynthesis, preferably oligonucleotide synthesis, which method employs a method for the preparation of a universal solid support as described herein.

Another aspect of the present invention pertains to an oligonucleotide which has been prepared using a method of oligosynthesis, preferably oligonucleotide synthesis, which method employs a universal solid support as described herein.

It will be apparent to the skilled artisan that particular features of individual aspects and embodiments of the invention will also pertain to other aspects and embodiments of the invention.

### DETAILED DESCRIPTION OF THE INVENTION

One aspect of the present invention pertains to methods for the preparation of a universal solid support for oligosynthesis, which support is useful, for example, in solid-phase oligonucleotide synthesis.

The term “oligosynthesis,” as used herein, pertains to the synthesis of oligomers or polymers, typically, though not exclusively, from monomer units, without reference to the composition of the oligomer or polymer. A preferred oligosynthesis is oligonucleotide synthesis.

The term “oligonucleotide synthesis,” as used herein, pertains to the synthesis of oligonucleotides, and analogs, mimics, and derivatives thereof, typically, though not exclusively, from monomer units (e.g., nucleoside phosphoramidites).

As used herein, the term “solid-phase oligonucleotide synthesis” is used in the conventional sense to describe synthetic methods in which the growing oligomer (e.g., oligonucleotide) is supported or otherwise attached (i.e., covalently linked) to a solid support, in contrast to the case for well-known “solution-phase oligonucleotide synthesis” methods.

Solid Support

The term “solid support” is used herein in the conventional sense, and refers to solid supports conventionally used in organic chemical synthesis, and more particularly, solid-phase oligonucleotide synthesis.

The solid support is typically formed from one or more insoluble or a substantially insoluble materials, which materials may be inorganic or organic, which materials are chemically inert (or substantially chemically inert) with respect to the reagents and conditions used during oligonucleotide synthesis (e.g., deprotection, coupling, oxidation, cleavage, and the like).

In one embodiment, the solid support is formed from an inorganic macroporous material. Examples of inorganic materials include, but are not limited to, silica, kieselguhr, porous glass, aluminosilicate, borosilicate, metal oxides (for example, aluminium oxide, nickel oxide, and iron oxide), and/or clay.

In one embodiment, the solid support is formed from an organic polymeric material, which polymeric material is optionally crosslinked. Examples of organic polymeric materials include, but are not limited to, cellulose, polysaccharide, crosslinked polysaccharides, polystyrene, crosslinked polystyrene, polyacryloylmorpholide, polyamide resin, polyacryloyl pyrollidone, polyethylene glycol, crosslinked polyethylene glycol, polyethylene glycol-polystyrene, cross-linked dextran, and/or cross-linked agarose.

In one embodiment, the solid support is formed from both an inorganic material and an organic polymeric material. Examples of such solid supports include, but are not limited to, acrylamide-kieselguhr resins, PEPSYN K resins, and POLYHIPE resins.

Commonly-used solid supports, which are suitable for use in the present invention, include but are not limited to, controlled pore glass beads (CPG) (for example, from Cruachem®), silica gel beads (for example, from Merck® or CPG, Inc.®), and polystyrene beads (for example, Porasil C®).

Other known solid supports which have been used in connection with oligonucleotide synthesis are also suitable for use in accordance with the present invention (see, for example, Gait, 1984 and Koester et al., 1990).

The solid support may be in any suitable form, including but not limited to, resins, particles, beads, fibres, films, and the like.

For use in the present invention, the solid support must be “functionalized,” that is, be in a form, or be derivatized to be in a form, in which one or more pendant functional groups are covalently linked to the solid support. These pendant functional groups permit attachment, synthesis, extension, and/or growth of a molecule to the solid support, as in, for example, solid phase oligonucleotide synthesis.

Examples of pendant functional groups include, but are not limited to, —NH₂, —OH, and —SH, also denoted herein as -J¹-H, wherein J¹ is —NH—, —O—, or —S—, respectively. In these cases, the solid support and the pendant functional group may be denoted as shown below. In a preferred embodiment, the pendant functional group is —NH₂.

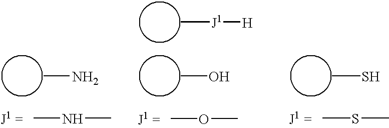

Examples of how solid supports may be functionalized are described in Gait et al., 1984. For example, in one embodiment, amino pendant functional groups may be linked to the solid support via siloxy groups, as shown below (see page 46 of Gait et al., 1984).

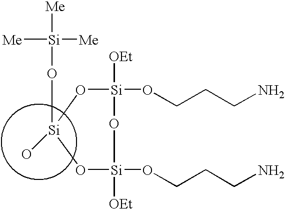

Pendant groups may be used to attach molecules via a covalent linkage using routine methods. Examples of such linkages include, but are not limited to, an amide linkage (—NH—CO—), an ester linkage (—O—CO—), and a thioester linkage (—S—CO—) (all denoted herein as -J¹-CO—).

The Linker Reagent and the Linker Group

The term “linker reagent,” as used herein, pertains to a compound of the formula:

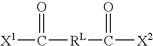

wherein X¹ and X² denote halogen atoms and are independently —F, —Cl, —Br, or —I, and R^{L} denotes a covalent bond or a divalent group. In one preferred embodiment, X¹ and X² are independently —Cl, —Br, or —I.

The term “pendant linker group,” as used herein, pertains to a monovalent moiety which is derived from a linker reagent, and which has the following formula:

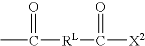

The term “linker group,” as used herein, pertains to a divalent moiety which is derived from a linker reagent, and which has the following formula:

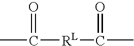

In one embodiment, as discussed below, R^{L} is a covalent bond.

In one embodiment, R^{L} denotes a divalent group which is an organic group comprising from 1 to 10 carbon atoms and from 0 to 5 heteroatoms selected from oxygen, nitrogen, and sulfur.

In one embodiment, R^{L} denotes a divalent C₁₋₁₀alkylene group. The term “divalent C₁₋₁₀alkylene group,” as used herein, pertains to divalent moieties obtained by removing one hydrogen atom from each of two different carbon atoms of a C₁₋₁₀alkane.

The term “C₁₋₁₀alkane,” as used herein, pertains to hydrocarbon compounds (compounds containing only hydrogen and carbon atoms) having from 1 to 10 carbon atoms, which compounds may be aliphatic or alicyclic, or a combination thereof, and which may be saturated, partially unsaturated, or fully unsaturated.

The term “aliphatic,” as used herein, pertains to groups which are linear or branched, but not cyclic. The term “alicyclic,” as used herein, pertains to groups which have one ring, or two or more rings (e.g., spiro, fused, bridged), but which are not aromatic. The term “saturated,” as used herein, pertains to groups which do not have any carbon-carbon double bonds or carbon-carbon triple bonds. The term “unsaturated,” as used herein, pertains to groups which have at least one carbon-carbon double bond or carbon-carbon triple bond.

Examples of saturated linear C₁₋₁₀alkanes include, but are not limited to, methane, ethane, n-propane, n-butane, n-pentane, and n-hexane.

Examples of saturated branched C₁₋₁₀alkanes include, but are not limited to, iso-butane, iso-pentane, neo-pentane, and iso-hexane.

Examples of saturated alicylic (carbocyclic) C₁₋₁₀alkanes (also referred to as “C₃₋₁₀cycloalkanes”) include, but are not limited to, cyclopropane, cyclobutane, cyclopentane, cyclohexane, as well as methylcyclopropane, methylcyclobutane, methylcyclopentane, and methylcyclohexane.

Examples of unsaturated C₁₋₁₀alkanes which have one or more carbon-carbon double bonds (also referred to as “C₂₋₇alkenes”) include, but are not limited to, ethene, propene, butene, and butadiene.

Examples of unsaturated C₁₋₁₀alkyanes which have one or more carbon-carbon triple bonds (also referred to as “C₂₋₇alkynes”) include, but are not limited to, ethyne, propyne, butyne, and butadiyne.

Examples of unsaturated alicylic (carbocyclic) C₁₋₁₀alkanes which have one or more carbon-carbon double bonds (also referred to as “C₃₋₁₀cycloalkenes”) include, but are not limited to, cyclopropene and cyclohexene, as well as groups which comprise such groups, including but not limited to methylcyclopropene and methylcyclohexene.

In one preferred embodiment, the C₁₋₁₀alkane is a saturated linear alkane of the formula CH₃(CH₂)ₙCH₃, wherein n is an integer from 1 to 8. Examples of such saturated linear C₁₋₁₀alkanes include, but are not limited to, methane, ethane, propane, n-butane, and n-pentane.

In one preferred embodiment, R^{L} has the formula —(CH₂)ₙ—, wherein n is an integer from 1 to 10. In one preferred embodiment, n is an integer from 1 to 8. In one preferred embodiment, n is an integer from 1 to 6. In one preferred embodiment, n is an integer from 1 to 4. In one preferred embodiment, n is an integer from 1 to 3. In one preferred embodiment, n is an integer from 1 to 2. Examples of such groups include, but are not limited to, methylene (n=1, —CH₂—), ethylene (n=2, —CH₂CH₂—), trimethylene (n=3, —CH₂CH₂CH₂—), tetramethylene (n=4, —CH₂CH₂CH₂CH₂—), and pentamethylene (n=5, —CH₂CH₂CH₂CH₂CH₂—).

In one preferred embodiment, R^{L} is —(CH₂)ₙ— wherein n is an integer from 0 to 10 (when n is 0, R^{L} is a covalent bond). In one preferred embodiment, n is an integer from 0 to 8. In one preferred embodiment, n is an integer from 0 to 6. In one preferred embodiment, n is an integer from 0 to 4. In one preferred embodiment, n is an integer from 0 to 3. In one preferred embodiment, n is an integer from 0 to 2. In one preferred embodiment, n is 0 or 1. In one preferred embodiment, n is 0, and R^{L} is a covalent bond.

In one preferred embodiment, the linker reagent has the formula:

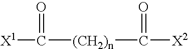

wherein X¹ and X² are independently —Cl, —Br, or —I, and n is an integer from 0 to 10, more preferably from 0 to 8, more preferably from 0 to 6, more preferably from 0 to 4, more preferably from 0 to 3, more preferably 0 to 2. In one preferred embodiment, n is 0 or 1. In one preferred embodiment, n is 0.

In one preferred embodiment, X¹ and X² are independently —Cl or —Br. In one preferred embodiment, X¹ and X² are the same. In one preferred embodiment, X¹ and X² are both —Cl or both —Br. In one preferred embodiment, X¹ and X² are both —Cl.

In one preferred embodiment, R^{L} is a covalent bond and the linker reagent has the structure X¹—CO—CO—X². Such compounds may be conveniently referred to as “oxalyl dihalides.” In one preferred embodiment, R^{L} is a covalent bond and X¹ and X² are the same. In one preferred embodiment, R^{L} is a covalent bond and X¹ and X² are both —Cl or both —Br. In one preferred embodiment, R^{L} is a covalent bond and X¹ and X² are both —Cl, and the linker reagent is as shown below, and is often referred to as oxalyl dichloride, shown below, which has a melting point of −16 EC and a boiling point of about 64 EC (1 atm).

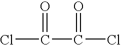

In one preferred embodiment, R^{L} is —CH₂— and the linker reagent has the structure X¹—CO—CH₂—CO—X². Such compounds may be conveniently referred to as “malonyl dihalides.” In one preferred embodiment, R^{L} is —CH₂— and X¹ and X² are the same. In one preferred embodiment, R^{L} is —CH₂— and X¹ and X² are both —Cl or both —Br. In one preferred embodiment, R^{L} is —CH₂— and X¹ and X² are both —Cl, and the linker reagent is as shown below, and is often referred to as malonyl dichloride, shown below, which has a boiling point of about 58 EC (26 mm Hg, 3.5 kPa).

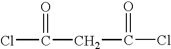

In one preferred embodiment, R^{L} is —CH₂— and the linker reagent has the structure X¹—CO—CH₂CH₂—CO—X². Such compounds may be conveniently referred to as “succinyl dihalides.” In one preferred embodiment, R^{L} is —CH₂CH₂— and X¹ and X² are the same. In one preferred embodiment, R^{L} is —CH₂CH₂— and X¹ and X²are both —Cl or both —Br. In one preferred embodiment, R^{L} is —CH₂CH₂— and X¹ and X² are both —Cl, and the linker reagent is as shown below, and is often referred to as succinyl dichloride, shown below, which has a melting point of 20 EC and a boiling point of about 194 EC (1 atm).

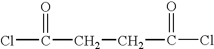

In one embodiment, R^{L} denotes a divalent group which is an organic group comprising from 1 to 10 carbon atoms and from 0 to 5 heteroatoms selected from oxygen, nitrogen, and sulfur, which divalent group, R^{L}, comprises one or more moieties selected from optionally substituted C₁₋₁₀alkylene groups and optionally substituted C₅₋₂₀arylene groups, which groups (if there are a plurality) are linked via a covalent bond or via a covalent linkage, and which divalent group, R^{L}, optionally includes a terminal covalent heteroatom linkage at one or both termini, in which case it may optionally form a larger covalent linkage with the adjacent carbonyl groups. Examples of covalent heteroatom linkages include, but are not limited to, an ether linkage (—O—) and a thioether linkage (—S—). When positioned at a terminus, these linkages form ester (—C(═O)O—) and thioester (—C(—O)S—) linkages, respectively.

The term “C₅₋₂₀arylene group,” as used herein, pertains to a divalent moiety obtained by removing one hydrogen atom from each of two different carbon atoms of an optionally substituted C₅₋₂₀arene.

In one embodiment, R^{L} denotes the following group, referred to as 2-(2-nitrophenyl)ethyl. See, for example, Eritja et al., 1991.

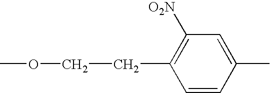

In one embodiment, R^{L} denotes the following group, referred to as Q-support. See, for example, Pon et al., 1997.

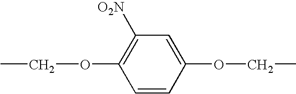

In one embodiment, R^{L} denotes the following group, referred to as 3′-alkylcarboxylic acid. See, for example, Tracy et al., 1997.

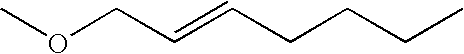

Methods of synthesis for the linker reagents are known in the art. Indeed, many of the linker reagents are commercially available.

Preferably, the linker reagent is selected to be a liquid at normal pressure (1 atm) and the relevant temperature, specifically, the temperature at which the step of reacting a pendant functional group of a solid support with a linker reagent is performed, and/or the temperature at which the step of removing at least a portion of excess unreacted linker reagent by evaporation is performed, preferably the latter.

Preferably, the linker reagent is selected to be a volatile compound at the relevant temperature. The term “volatile,” as used herein, describes a compound (e.g., a linker reagent) which has a substantial vapour pressure at the relevant temperature, preferably at least 10 Pa (−0.075 mm Hg), more preferably at least 100 Pa (˜0.75 mm Hg), more preferably at least 1000 Pa (˜7.5 mm Hg).

More preferably, the linker reagent is selected to be a volatile liquid at the relevant temperature.

Cyclic Reagent

The term “cyclic reagent,” as used herein, pertains to a compound of the formula:

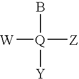

wherein:

Q denotes a cyclic group;

B denotes a base group;

W denotes a reactive conjugating group;

Y denotes an oligosynthesis group; and,

Z, if present, denotes an auxiliary group.

Cyclic Group, Q

The term “cyclic group,” as used herein, pertains to a moiety which has a single non-aromatic ring, which ring has from 5 to 7 ring atoms, which ring atoms are (a) all carbon atoms (in which case the ring may be referred to as a “carbocyclic ring”), or (b) carbon atoms and one or two heteroatoms selected from oxygen, nitrogen, and sulfur (in which case the ring may be referred to as a “heterocyclic ring”).

The ring bonds, which join the ring atoms, may be single, double, or triple bonds, but the ring itself must not be aromatic. In one preferred embodiment, the ring bonds are single or double bonds. In one preferred embodiment, the ring bonds are all single bonds.

In one preferred embodiment, the cyclic group has a single non-aromatic ring, which ring has 5 or 6 ring atoms, which ring atoms are (a) all carbon atoms or (b) carbon atoms and one or two heteroatoms selected from oxygen, nitrogen, and sulfur, and all ring bonds are single bonds.

In one preferred embodiment, the cyclic group has a single non-aromatic ring, which ring has 5 or 6 ring atoms, which ring atoms are (a) all carbon atoms or (b) carbon atoms and one oxygen atom, and all ring bonds are single bonds.

In one preferred embodiment, the cyclic group has a single non-aromatic ring, which ring has 5 ring atoms, which ring atoms are (a) all carbon atoms or (b) carbon atoms and one oxygen atom, and all ring bonds are single bonds.

In one preferred embodiment, the cyclic group has a single non-aromatic ring, which ring has 6 ring atoms, which ring atoms are (a) all carbon atoms or (b) carbon atoms and one oxygen atom, and all ring bonds are single bonds.

In one preferred embodiment, the cyclic group has a single non-aromatic ring, which ring has 5 ring atoms, which ring atoms are all carbon atoms, and all ring bonds are single bonds. In this case, the ring is a cyclopentane ring.

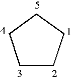

In one preferred embodiment, the cyclic group has a single non-aromatic ring, which ring has 6 ring atoms, which ring atoms are all carbon atoms, and all ring bonds are single bonds. In this case, the ring is a cyclohexane ring.

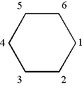

In one preferred embodiment, the cyclic group has a single non-aromatic ring, which ring has 5 ring atoms, which ring atoms are carbon atoms and one oxygen atom, and all ring bonds are single bonds. In this case, the ring is a tetrahydrofuran ring.

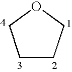

In one preferred embodiment, the cyclic group has a single non-aromatic ring, which ring has 6 ring atoms, which ring atoms are carbon atoms and one oxygen atom, and all ring bonds are single bonds. In this case, the ring is a tetrahydropyran ring.

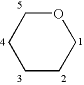

The base group, B, the reactive conjugating group, W, the oligosynthesis group, Y, and the auxiliary group, Z, if present, are each separately attached to a carbon ring atom of the cyclic group, Q, either directly (via a covalent bond), or indirectly (via an intermediate covalent linkage). Examples of intermediate covalent linkages include, but are not limited to, —(CH₂)ₙ—, wherein n is an integer from 1 to 4, for example, —CH₂— and —CH₂CH₂—. In one preferred embodiment, these groups are attached directly.

In one preferred embodiment, the oligosynthesis group, Y, and the auxiliary group, Z, which is present, are each directly attached to vicinal carbon ring atoms of the cyclic group, Q. In one preferred embodiment, these groups are attached with a cis orientation.

The cyclic group may also have other optional substituents, that is, substituents other than B, W, Y, and Z. Examples of such optional substituents include, but are not limited to, C₁₋₇alkyl groups, C₁₋₇alkoxy groups, and acyl groups.

The Base Group, B

The base group, B, is a derived from a nucleic acid base. The terms “nucleic acid base,” “nucleotide base,” and “nucleoside base” are used herein in the conventional sense to refer to purine and pyrimidine bases, and derivatives and analogs thereof, such as those typically found in nucleic acids.

Preferred nucleic acid bases include the well-known naturally occurring purines: adenine and guanine; and pyrimidines: cytosine, thymine, and uracil. Other examples of the nucleic acid bases known in the art include purine and pyrimidine derivatives and analogs, including but not limited to, sarcine (also known as hypoxanthine or sarkin), aziridinylcytosine, 4-acetylcytosine, 5-fluorouracil, 5-bromouracil, 5-carboxymethylaminomethyl-2-thiouracil, 5-carboxymethylaminomethyluracil, N⁶-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxyuracil, 2-methylthio-N⁶-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, 5-pentynyluracil and 2,6-diaminopurine.

In one preferred embodiment, the base group, B, is derived from a nucleic acid base selected from the group consisting of: adenine, guanine, cytosine, thymine, uracil, and sarcine (the base associated with the nucleoside inosine), or a protected form thereof. In one preferred embodiment, the base moiety, B, is derived from sarcine, or a protected form thereof.

The base moiety, B, for which the ring atoms are conventionally numbered with unprimed numbers, is preferably covalently attached to the cyclic reagent via a ring atom of the base. The base is preferably attached via a ring nitrogen atom of the base. For pyrimidines, attachment at the (N)-1-position is preferred, whereas for purines, attachment at the (N)-9 position is preferred.

In one preferred embodiment, the base group, B, is selected from the following, or a protected form thereof:

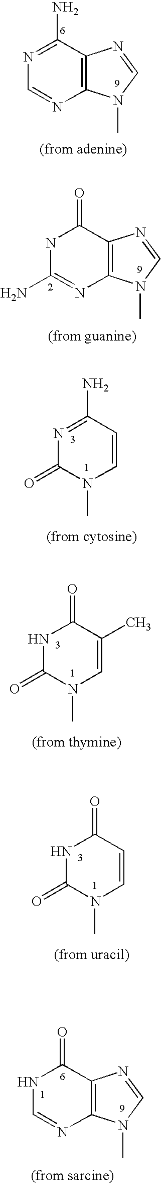

In one preferred embodiment, the base group, B, is the following, or a protected form thereof:

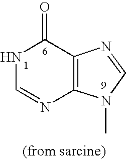

The reactive functional groups of the base group, B, may be in protected or unprotected forms. Protected forms of such functional groups are well known in the art. For example, primary amines (—NH₂), such as those found in adenine, guanine, and cytosine, may be protected as an amide, for example, as isobutyramide or benzamide. Such protection is often employed during oligonucleotide synthesis.

The term “protected,” as used herein, refers to functional groups which are essentially unreactive towards other available functional groups under specified conditions. The term “functional group protection” is used herein in the conventional chemical sense to refer to common chemical methods employed to reversibly render unreactive a functional group, which otherwise would be reactive, under specified conditions (such as pH, temperature, radiation, solvent, and the like). A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

Reactive Conjugating Group, W

The term “reactive conjugating group,” as used herein, refers to a functional group which reacts with the —C(═O)X² group of the linker reagent to form a covalent linkage, and thereby form a conjugate therewith.

In a preferred embodiment, the reactive conjugating group, W, is -J²-H, wherein J² is —O—, —NH—, or —S—, that is, the reactive conjugating group, W, is —OH, —NH₂, or —SH, respectively. In this case, the cyclic reagent has the following formula:

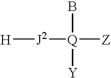

In one preferred embodiment, the reactive conjugating group is —OH or —NH₂. In one preferred embodiment, the reactive conjugating group is —OH.

When the reactive conjugating group is -J²-H, the covalent linkage formed upon reaction with —C(═O)X² is —C(═O)-J², that is, —C(═O)—O—, —C(═O)—NH—, and —C(═O)—S—, respectively.

As discussed below, the reactive conjugating group may be activated, for example, by the presence of a base such as N-methylimidazole (NMI) and/or 4-dimethylaminopyridine (DMAP). Such activation facilitates and/or accelerates the reaction between the reactive conjugating group and the —C(═O)X² group of the linker reagent.

Oligosynthesis Group, Y

The term “oligosynthesis group,” as used herein, refers to a functional group, or a protected functional group, which facilitates oligosynthesis, thatis, a group which, when in a deprotected and/or derivatized form, will react with a suitable monomer to initiate growth of desired oligomer.

In one embodiment, the oligosynthesis group, Y, is —OH, —NH₂, or —SH, or a protected form thereof. In one embodiment, the oligosynthesis group, Y, is —OH or a protected form thereof.

In one embodiment, the oligosynthesis group, Y, is —OH, —NH₂, or —SH in a protected form, and which is deprotected under the deprotection conditions used in oligosynthesis. For example, the group may be deprotected under the conditions used in the deprotection step in conventional oligonucleotide (e.g., phosphoramidite) synthesis cycle, and thus be available as the anchor for attachment of the first nucleotide. Typically and preferably, the deprotection step employs acidic conditions.

Examples of suitable protecting groups, R, which protect —OH as —OR, include, but are not limited to:

t-butyl (tBu);

4-dimethoxytrityl(MMT);

4,4′-dimethoxytrityl(DMT);

4,4′,4″-tris-(benzyloxy)trityl(TBTr);

4,4′,4″-tris-(4,5-dichlorophtalimido)trityl(CPTr)

4,4′,4″-tris-(levulinyloxy)trityl(TLTr);

3-(imidazolylmethyl)-4-4′-dimethoxytrityl(IDTr)

9-phenylxanthen-9-yl(pixyl);

9-(p-methoxyphenyl)xanthen-9-yl(Mox);

4-decyloxytrityl(C₁₀Tr);

4-hexadecyloxytrityl(C₁₆Tr);

9-(4-octadecyloxyphenyl)xanthene-9-yl(C₁₈Px); and,

1,1-bis-(4-methoxyphenyl)-1′-pyrenyl methyl(BMPM).

Examples of suitable protecting groups, —C(═O)OR, which protect —NH₂ as —NH—C(═O)—OR, include, but are not limited to:

tert-butoxy carbonyl (—C(═O)—OC(CH₃)₃, t-BOC); and,

benzyloxy carbonyl (—C(═O)—OCH₂Ph, CBZ).

These and other suitable protecting groups are described, for example, in Beaucage et al., 1992.

Auxiliary Group, Z

The term “auxiliary group,” as used herein, refers to a group which is optional, and which, if present, is a functional group or, more preferably, a protected functional group. In one embodiment, the auxiliary group is protected and is stable during conventional (e.g., phosphoramidite) oligonucleotide synthesis, and, following synthesis, is deprotected concurrently with cleavage, that is, under conventional cleavage conditions. Thus, the auxiliary group, in protected form, is typically different from the oligosynthesis group, in protected form, since the former is stable during oligosynthesis and the latter is deprotected, or in a deprotected form, during one or more steps of oligosynthesis.

The auxiliary group, Z, may, in protected or deprotected form, interact with, and/or form a functional group together with, the oligosynthesis group, Y.

In one embodiment, the auxiliary group, Z, is —OH, —NH₂, or —SH, or a protected form thereof. In one preferred embodiment, the auxiliary group is —OH or a protected form thereof.

In one preferred embodiment, both the auxiliary group, Z, and the oligosynthesis group, Y, in deprotected form, are —OH, and, in protected form, together form part of a cyclic structure with an O—O-methoxyethylidene group, shown below:

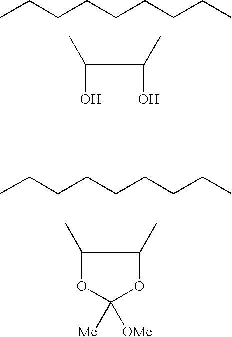

In one preferred embodiment, the auxiliary group, in deprotected form, is —OH, and in protected form is —OR, wherein R is an acyl group, a C₁₋₇alkyl group, or a silyl group.

In one preferred embodiment, the auxiliary group, in deprotected form, is —OH, and in protected form is —OR, wherein R is an acyl group.

The term “acyl group,” as used herein, pertains to a moiety of the formula —C(═O)R, wherein R is an acyl substituent, for example, a C₁₋₇alkyl group (to give a group referred to as C₁₋₇alkylacyl), a C₃₋₂₀heterocyclyl group (to give a group referred to as C₃₋₂₀heterocyclylacyl), or a C₅₋₂₀aryl group (to give a group referred to as C₅₋₂₀arylacyl), preferably a C₁₋₇alkyl group. Examples of acyl groups include, but are not limited to, —C(═O)CH₃ (acetyl), —C(═O)CH₂CH₃ (propionyl), —C(═O)CH₂CH₂CH₃ (n-butanoyl), —C(═O)C(CH₃)₃ (t-butanoyl), and —C(═O)Ph (benzoyl).

The term “C₁₋₇alkyl group,” as used herein, pertains to monovalent alkyl groups having from 1 to 7 carbon atoms, which may be aliphatic or alicyclic, or a combination thereof, and which may be saturated, partially unsaturated, or fully unsaturated.

Examples of saturated linear C₁₋₇alkyl groups include, but are not limited to, methyl, ethyl, n-propyl, n-butyl, and n-pentyl (amyl).

Examples of saturated branched C₁₋₇alkyl groups include, but are not limited to, iso-propyl, iso-butyl, sec-butyl, tert-butyl, and neo-pentyl.

Examples of saturated alicylic (carbocyclic) C₁₋₇alkyl groups (also referred to as “C₃₋₇cycloalkyl” groups) include, but are not limited to, cyclopropyl, cyclobutyl, cyclopentyl, and cyclohexyl, as well as groups which comprise such groups, including, but not limited to, cyclopropylmethyl and cyclohexylmethyl.

Examples of unsaturated C₁₋₇alkyl groups which have one or more carbon-carbon double bonds (also referred to as “C₂₋₇alkenyl” groups) include, but are not limited to, ethenyl (vinyl) and 2-propenyl (allyl).

Examples of unsaturated C₁₋₇alkyl groups which have one or more carbon-carbon triple bonds (also referred to as “C₂₋₇alkynyl” groups) include, but are not limited to, ethynyl (ethinyl) and 2-propynyl (propargyl).

Examples of unsaturated alicylic (carbocyclic) C₁₋₇alkyl groups which have one or more carbon-carbon double bonds (also referred to as “C₃₋₇cycloalkenyl” groups) include, but are not limited to, cyclopropenyl and cyclohexenyl, as well as groups which comprise such groups, including but not limited to cyclopropenylmethyl and cyclohexenylmethyl.

The term “C₃₋₂₀heterocyclyl,” as used herein, pertains to a monovalent moiety obtained by removing a hydrogen atom from a ring atom of an alicyclic (i.e., non-aromatic cyclic) compound, said compound having one ring, or two or more rings (e.g., spiro, fused, bridged), having from 3 to 20 ring atoms, of which from 1 to 10 are ring heteroatoms, including, but not limited to, nitrogen, oxygen, and sulfur. Preferably, each ring has from 3 to 7 ring atoms, of which from 1 to 4 are ring heteroatoms. “C₃₋₂₀” denotes ring atoms, whether carbon atoms or heteroatoms.

Examples of C₃₋₂₀heterocyclyl groups having one nitrogen ring atom include, but are not limited to, those derived from aziridine, azetidine, pyrrolidine, pyrroline, pyrrolinine, piperidine, dihydropyridine, and tetrahydropyridine.

Examples of C₃₋₂₀heterocyclyl groups having one oxygen ring atom include, but are not limited to, those derived from oxirane, oxetane, oxolane (tetrahydrofuran), oxole (dihydrofuran), oxane (tetrahydropyran), dihydropyran, and pyran.

Examples of C₃₋₂₀heterocyclyl groups having one sulfur ring atom include, but are not limited to, those derived from thiolane and tetrahydrothiopyran.

Examples of C₃₋₂₀heterocyclyl groups having two oxygen ring atoms include, but are not limited to, those derived from dioxane.

Examples of C₃₋₂₀heterocyclyl groups having two nitrogen ring atoms include, but are not limited to, those derived from imidazolidine, imidazoline, and piperazine.

Examples of C₃₋₂₀heterocyclyl groups having one nitrogen ring atom and one oxygen ring atom include, but are not limited to, those derived from tetrahydrooxazole, dihydrooxazole, tetrahydroisoxazole, dihydroiosoxazole, morpholine, tetrahydrooxazine, and dihydrooxazine.

Examples of C₃₋₂₀heterocyclyl groups having one oxygen ring atom and one sulfur ring atom include, but are not limited to, oxathiolane and oxathiane.

The term “C₅₋₂₀aryl,” as used herein, pertains to a monovalent moiety obtained by removing a hydrogen atom from a ring atom of an aromatic compound (a C₅₋₂₀arene), said compound having one ring, or two or more fused rings, and having from 5 to 20 ring atoms. The ring atoms may be all carbon atoms, as in “carboaryl groups,” or may include one or more heteroatoms (including but not limited to oxygen, nitrogen, and sulfur), as in “heteroaryl groups.” In the latter case, the group may conveniently be referred to as a “C₅₋₂₀heteroaryl” group, wherein “C₅₋₂₀” denotes ring atoms, whether carbon atoms or heteroatoms. Preferably, each ring has from 3 to 7 ring atoms, of which from 0 to 4 are ring heteroatoms.

Examples of C₅₋₂₀aryl groups which do not have heteroatoms (i.e., (carboaryl groups) include, but are not limited to, phenyl, naphthyl, anthracenyl, phenanthrenyl, and pyrene.

Examples of C₅₋₂₀heteroaryl groups include, but are not limited to, pyrrolyl, imidazolyl, pyrazolyl, triazolyl, pyridyl, pyrazinyl, pyrimidinyl, pyridazinyl, triazinyl, furanyl, thienyl, thiazolyl, isothiazolyl, pyranyl, pyronyl, benzopyronyl, oxazolyl, isoxazolyl, oxadiazolyl, oxatriazolyl, oxathiazolyl, and oxathiazinyl.

The term “silyl group,” as used herein, pertains to a moiety of the formula —SiR₃, where R is a silyl substituent, for example, —H, a C₁₋₇alkyl group, a C₃₋₂₀heterocyclyl group, or a C₅₋₂₀aryl group, preferably —H, a C₁₋₇alkyl group, or a C₅₋₂₀aryl group. Examples of silyl groups include, but are not limited to, —SiH₃, —SiH₂(CH₃), —SiH(CH₃)₂, —Si(CH₃)₃, —Si(t-Bu)(CH₃)₂, —Si(i-Pr)₃, and —Si(t-Bu)₃.

Examples of Cyclic Reagents

In one preferred embodiment, the oligosynthesis group, Y, and the optional auxiliary group, Z, are linked directly to cyclic group, Q, by a covalent bond, and are positioned “vicinal” (i.e., Y and Z are attached to adjacent carbon ring atoms of the cyclic group, Q). Examples of cyclic moieties, Q, with vicinally attached Y and Z groups, include, but are not limited to, the following.

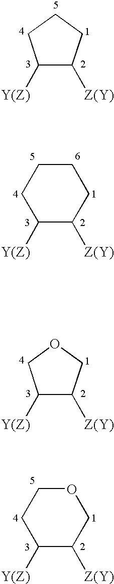

The notation “Y(Z)” and “Z(Y)” indicates all four of the possibilities: 3′-Y-2′-Z; 3′-Z-2′-Y; 3′-Y-2′-H (where Z is absent, i.e., Z is —H); and 3′-H-2′-Y (where Z is absent, i.e., Z is —H).

In one preferred embodiment, the Y and Z groups are also positioned “cis.” Examples of such cyclic moieties, Q, with cis-vicinally attached Y and Z groups (both “down,” below the ring), include, but are not limited to, the following:

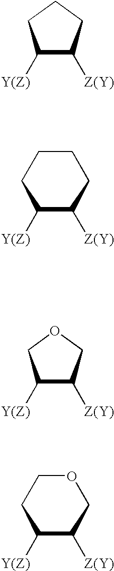

In one preferred embodiment, the W group is covalently linked to the cyclic group, Q, via a —CH₂— group, and the B group is directly linked to the cyclic moiety, Q. Examples of such cyclic reagents include, but are not limited to, the following:

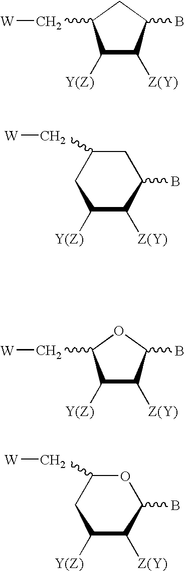

In one embodiment, the cyclic reagent resembles a monosaccharide sugar. Sugars, in cyclic form, are conventionally named according to the number of ring atoms. For example, a furanose has a 5-membered ring and a pyranose has a 6-membered ring. Sugars are also conventionally named according to the overall number of carbon atoms. For example, a pentose has 5 carbon atoms and a hexose has 6 carbon atoms. Sugars, in cyclic form, are further conventionally named using prefixes, such as ribo-, lyxo-, xylo-, galacto-, sucro-, fructo-, and arabino-, according both to number of carbon atoms and the orientation of the ring substituents. Furthermore, sugars may be in any of their enantiomeric, diasteriomeric or stereoisomeric forms (e.g., D-, L-, ∀-, ∃-, and combinations thereof).

In one preferred embodiment, the cyclic reagent resembles ∃-D-ribofuranose (also referred to as ∃-D-ribose), 2′-deoxy-∃-D-ribofuranose (also referred to as 2′-deoxy-∃-D-ribose), or 3′-deoxy-∃-D-ribofuranose (also referred to as 3′-deoxy-∃-D-ribose), which have the following structures:

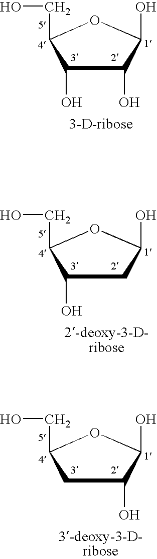

Thus, in one preferred embodiment, the cyclic reagent has the following structure. When Z is present, the compound is a ∃-D-ribonucleoside. When Z is absent (i.e., Z is —H), the compound is a 2′-deoxy-∃-D-ribonucleoside.

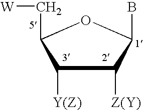

In one preferred embodiment, W is —OH, Y is —OH or a protected form thereof, and Z, if present, is —OH or a protected form thereof, for example, as shown in the following structures:

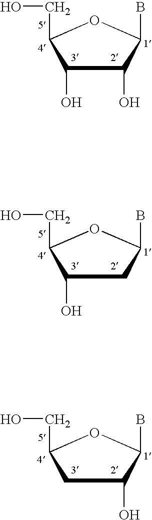

In one preferred embodiment, the cyclic reagent is inosine, shown below, or a protected form thereof.

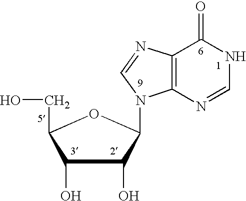

In one preferred embodiment, the cyclic reagent is the inosine derivative, 9-∃-D-(2′-3′-O-O-methoxyethylidene-ribofuranosyl)hypoxanthine, shown below, in which the 2′- and 3′-hydroxyl groups have been jointly protected as an O—O-methoxyethylidene group.

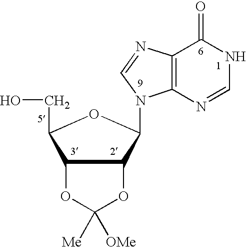

Reaction Apparatus

As mentioned above, one step of the preparation of a solid support suitable for use in solid-phase oligosynthesis involves: removing at least a portion of excess unreacted linker reagent by evaporation, as may be achieved, for example, by using an evaporation apparatus.

Thus, in one embodiment, one or more steps of the method is performed using (i.e., within) an evaporation apparatus. In one embodiment, at least the step of removing at least a portion of excess unreacted linker reagent by evaporation is performed using an evaporation apparatus.

In one embodiment, at least the step of reacting a pendant functional group of a solid support with a linker reagent, and the step of removing at least a portion of excess unreacted linker reagent by evaporation, are performed using an evaporation apparatus.

In one embodiment, at least the steps of reacting a pendant functional group of a solid support with a linker reagent, removing at least a portion of excess unreacted linker reagent by evaporation, and reacting said pendant linker group with a cyclic reagent, are performed using an. evaporation apparatus.

In another embodiment, all of the steps of the method are performed using an evaporation apparatus.

The term “evaporation apparatus,” as used herein, pertains to any apparatus suitable for carrying out chemical reactions, such as those in the steps of the present method, which facilitates both airtight anhydrous conditions (e.g., a sealed environment under a dry atmosphere, such as dry air, dry nitrogen, dry argon) and evaporative removal of one or more reaction mixture components (e.g., reagent, product, by-product, solvent, and the like), particularly excess linker reagent.

Examples of suitable apparati include, but are not limited to, a rotavapor apparatus and a distillation apparatus. In one preferred embodiment, the apparatus is a rotavapor apparatus, for example, Model R-114, from Büchi®.

Reaction of Support with Linker Reagent

As mentioned above, one step of the preparation of a solid support suitable for use in solid-phase oligosynthesis involves an “activating” step: reacting a pendant functional group of a solid support with a linker reagent, thereby forming a pendant linker group.

Examples of this reaction are shown below.

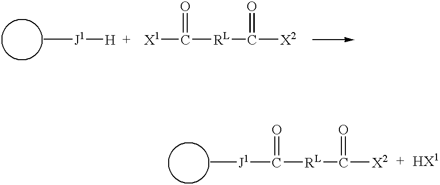

In general, the linker reagent is reactive towards water, and this is particularly true for the oxalyl dihalides, where R^{L} is a covalent bond. Consequently, in order to avoid degradation of the linker reagent, the activating step is preferably performed under anhydrous conditions, for example, in a sealed, airtight environment, under an dry atmosphere (e.g., dry air, dry nitrogen, dry argon), as may be achieved, for example, by using an evaporation apparatus. Higher yields are obtained using more anhydrous conditions.

As mentioned above, one step of the preparation of a solid support suitable for use in solid-phase oligosynthesis involves: removing at least a portion of excess unreacted linker reagent by evaporation.

The term “evaporation,” as used herein, pertains to the removal of a reaction mixture component (e.g., reagent, product, by-product, solvent, and the like), from the desired product, as a gas or vapour, via the gas or vapour phase.

Preferably, the linker reagent is selected to be a volatile compound, more preferably a volatile liquid, at the relevant temperature. When the linker reagent is so selected, excess linker reagent may be easily removed from the reaction mixture by evaporation, for example, by the application of reduced pressure and/or increased temperature. In this way, no additional purification is required, and this provides a substantial advantage over methods which require a separate, intensive, and often time consuming, purification step, to remove excess activating agent (e.g., linker reagent) which would otherwise lead to undesired side-reactions and by-products. For example, prior art methods invariably require a separate purification step between activation of the solid support and subsequent reaction with an initiating compound (e.g., cyclic reagent), or between activation of an initiating compound and subsequent reaction with the solid support.

Thus, in one embodiment, the evaporation is achieved by the application of reduced pressure and/or increased temperature. In one preferred embodiment, the evaporation is achieved by the application of reduced pressure. In one preferred embodiment, the evaporation is achieved by the application of increased temperature.

In one embodiment, the step of removing at least a portion of excess unreacted linker reagent is achieved solely by evaporation, that is, by evaporation and without the use of other non-evaporative methods of purification, such as filtration, blotting, washing (e.g., with one or more solvents or solvent mixtures), drying (e.g., over MgSO₄), solvent partitioning (e.g., between two or more solvents or solvent mixtures), trituration, or chromatography (e.g., column chromatography).

In one embodiment, the step of removing at least a portion of excess unreacted linker reagent by evaporation (or solely by evaporation) involves removing a substantial portion of excess unreacted linker reagent, preferably at least 50% of excess unreacted linker, more preferably at least 60%, more preferably at least 70%, more preferably at least 80%, more preferably at least 90%.

In one embodiment, the step of removing at least a portion of excess unreacted linker reagent is achieved by evaporation (or solely by evaporation), and, additionally, without the addition of a solvent or co-solvent (e.g., pyridine, methanol), as used, for example, in co-evaporation methods.

In one embodiment, the step of removing at least a portion of excess unreacted linker reagent is achieved by evaporation (or solely by evaporation), and, additionally, without the use of a desiccant (e.g., P₂O₅), as used, for example, in desiccation methods.

Preferably, the solid support is substantially dry before use, as may be achieved, for example, by drying in an oven (e.g., 30 min at 100 EC).

Suitable relative amounts of reagents and reaction conditions (e.g., temperature, time, and the like) may readily be determined by the skilled artisan.

In one preferred embodiment, from about 100 to about 1500 mn of linker compound is used for each 100 g of solid support, more preferably from about 200 to about 1000 mL of linker compound, more preferably from about 300 to about 800 mL of linker compound.

In one preferred embodiment, the reaction is carried out at a reaction temperature of from about 0 E to about 100 EC, more preferably from about 20 E to about 100 EC.

In one preferred embodiment, the reaction is carried out for a reaction time of from about 1 to about 240 min, more preferably from about 5 to about 120 min.

Optional Capping

As mentioned above, the preparation of a solid support suitable for use in solid-phase oligosynthesis optionally includes a “capping step” for pendant functional groups.

Thus, in one embodiment, the method further comprises, after said step of reacting a pendant functional group of a solid support with a linker reagent, or, after said step of removing at least a portion of excess unreacted linker reagent by evaporation, a step of:

(i) reacting at least a portion of any remaining pendant functional groups of said solid support with a capping reagent, thereby forming pendant capped functional groups.

Following the reaction of the pendant functional group of a solid support with a linker reagent, an optional “capping” step may be performed, in which “unused” pendant functional groups (i.e., pendant functional groups which have not reacted with a linker reagent) are converted to a protected or otherwise unreactive form.

For example, where the pendant functional group is —NH₂, acetic anhydride (Ac₂O, CH₃C(═O)—O—C(═O)CH₃) may be used, as shown in the following reaction.

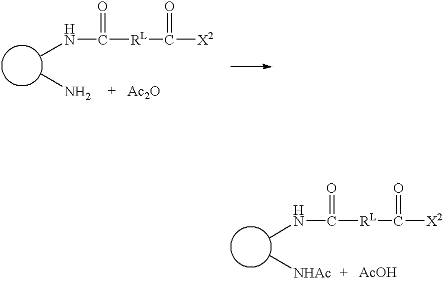

Other suitable capping reagents and conditions are well known in the art, and may be used where appropriate.

Where the pendant functional group is —OH, examples of suitable capping reagents include, but are not limited to, anhydrides and esters.

Where the pendant functional group is —SH, examples of suitable capping reagents include, but are not limited to, haloacetamides.

In one preferred embodiment, the capping reagent is selected to be a volatile liquid at, or near to, room temperature, so that excess capping reagent, and possibly capping by-products, can also be removed by evaporation, as described above for the excess linker reagent.

Suitable relative amounts of reagents and reaction conditions (e.g., temperature, time, and the like) may readily be determined by the skilled artisan.

In one preferred embodiment, from about 10 to about 200 mL of acetic anhydride, as capping reagent, and from about 1 to about 20 mL of N-methylimidazole (NMI) and/or from about 0.5 to about 2 g of 4-dimethylaminopyridine (DMAP) is used for each 100 g of solid support. In one preferred embodiment, from about 20 to about 100 mL of acetic anhydride, as capping reagent, and from about 2 to about 10 mL of N-methylimidazole (NMI) and/or about 1 g of 4-dimethylaminopyridine (DMAP) is used for each 100 g of solid support.

In one preferred embodiment, the reaction is carried out at a reaction temperature of from about 0 E to about 60 EC, more preferably from about 20 E to about 40 EC.

In one preferred embodiment, the reaction is carried out for a reaction time of from about 1 to about 60 min, more preferably from about 2 to about 30 min.

Reaction of Linker with Cyclic Reagent

As mentioned above, one step of the preparation of a solid support suitable for use in solid-phase oligosynthesis involves a “coupling” step: reacting the pendant linker group with a cyclic reagent, thereby forming a pendant cyclic group.

Examples of this reaction, wherein the reactive conjugating group, W, is -J²-H and J² is —NH—, —O—, or —S— (i.e., W is —NH₂, —OH, or —SH) are shown below.

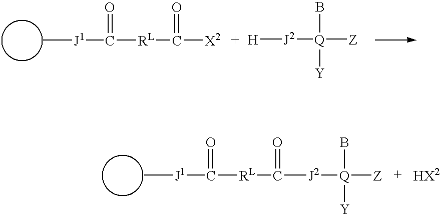

The reactive conjugating group may be activated, for example, by the presence of a base such as N-methylimidazole (NMI) and/or 4-dimethylaminopyridine (DMAP). Such activation facilitates and/or accelerates the reaction between the reactive conjugating group and the —C(═O)X² group of the linker reagent. This base may also combine with the HX² (e.g., HCl, HBr, HI) liberated by reaction with the —C(═O)X² group of the linker reagent. Examples of such bases include, but are not limited to, aqueous NaOH, aqueous KOH, pyridine, dimethylaminopyridine (DMAP), and N-methylimidazole (NMI).

Suitable relative amounts of reagents and reaction conditions (e.g., temperature, time, and the like) may readily be determined by the skilled artisan.

In one preferred embodiment, from about 5 to about 8 g of cyclic reagent in from about 200 to about 600 mL of pyridine, and from about 20 to about 60 mL of N-methylimidazole (NMI) is used for each 100 g of solid support. In one preferred embodiment, from about 6 to about 7 g of cyclic reagent in from about 300 to about 500 mL of pyridine, and from about 30 to about 50 mL of N-methylimidazole (NMI) is used for each 100 g of solid support.

In one preferred embodiment, the reaction is carried out at a reaction temperature of from about 0 E to about 60 EC, more preferably from about 20 E to about 40 EC.

In one preferred embodiment, the reaction is carried out for a reaction time of from about 5 to about 60 min, more preferably from about 10 to about 30 min.

Optional Capping

As mentioned above, the preparation of a solid support suitable for use in solid-phase oligosynthesis optionally includes a “capping step” for pendant linker groups.

Thus, in one embodiment, the method further comprises, after said step of reacting said pendant linker group with a cyclic reagent, a step of:

(i) reacting at least a portion of any remaining pendant linker groups of said solid support with a capping reagent, thereby forming pendant capped linker groups.

Following the reaction of the pendant linker groups with a cyclic reagent, an optional “capping” step may be performed, in which “unused” pendant linker groups (i.e., pendant linker groups which have not reacted with a cyclic reagent) are converted to a protected or otherwise unreactive form.

Examples of suitable capping reagents and conditions are described above.

Optional Purification

As mentioned above, the preparation of a solid support suitable for use in solid-phase oligosynthesis optionally includes a step of: removing at least a portion of excess unreacted cyclic reagent, capping reagent (if applicable), and by-products thereof.

Any suitable method may be used to effect this removal. In one preferred embodiment, the removal is achieved by filtration, for example, using a fritted glass funnel, for example, ROBU-POR-2® from Elvetec®.

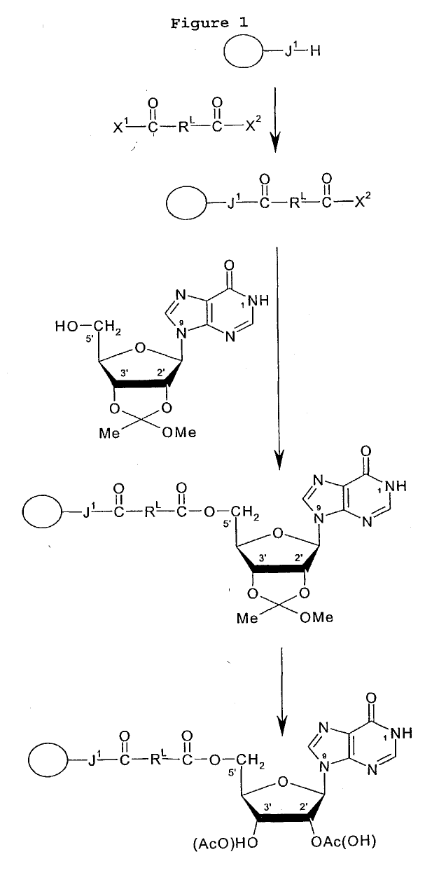

A chemical scheme illustrating the preparation of a preferred class of universal solid supports of the present invention is shown in FIG. 1, including an optional final deprotection step, which is typically the first step of oligonucleotide synthesis.

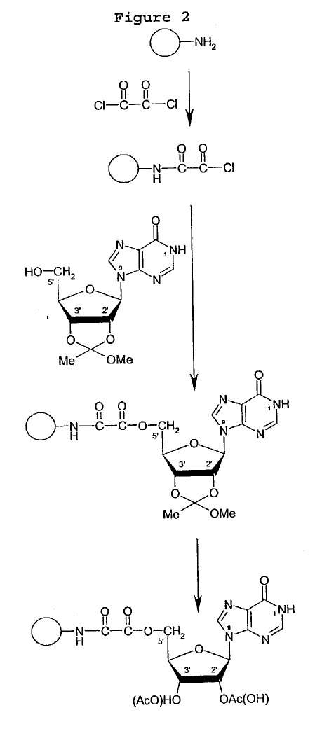

A chemical scheme illustrating the preparation of the universal solid support described in the Example is shown in FIG. 2, including an optional final deprotection step, which is typically the first step of oligonucleotide synthesis.

Universal Solid Supports

As mentioned above, one aspect of the present invention pertains to universal solid supports, for example, as prepared by the methods described herein.

In one embodiment, the universal solid support has the following formula:

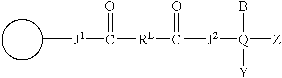

wherein J¹, J², R^{L}, Q, B, Y, and Z are as defined herein.

In one preferred embodiment, the universal solid support is the following, or a protected form thereof, wherein J¹ and R^{L} are as defined herein, or more preferably, J¹ is —NH— and R^{L} is —(CH₂)ₙ—, wherein n is an integer from 0 to 10:

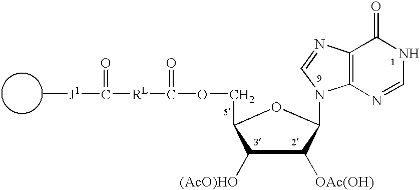

In one preferred embodiment, the universal solid support is the following, wherein J¹ and R^{L} are as defined herein, or more preferably, J¹ is —NH— and R^{L} is —(CH₂)ₙ—, wherein n is an integer from 0 to 10:

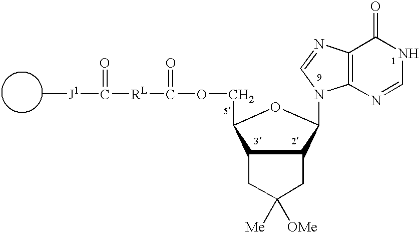

In one preferred embodiment, the universal solid support is the following, or a protected form thereof:

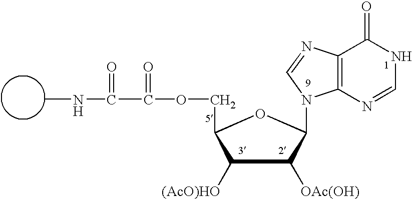

In one preferred embodiment, the universal solid support is the following:

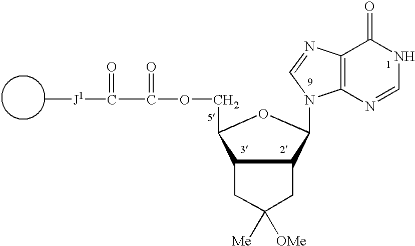

Oligonucleotides

As used herein, the terms “oligonucleotide” and “polynucleotide” are used interchangeably in the conventional sense to refer to molecules comprising two (hence 2-mer) or more (hence n-mer) nucleosides, each nucleoside being linked to at least one other nucleoside by an internucleoside linkage. The term “nucleoside” is used herein in the conventional sense and generally refers to compounds comprising a “sugar” moiety (e.g., ribose, 2′-deoxyribose) linked to a “nucleic acid base” (e.g., purines, pyrimidines). Examples of well-known nucleosides formed from ribose and adenine, guanine, uracil, and cytosine are, respectively, the “ribonucleosides”: adenosine, guanosine, uridine, and cytidine. Examples of well-known nucleosides formed from deoxyribose and adenine, guanine, thymine, and cytosine are respectively, the “deoxyribonucleosides”: deoxyadenosine, deoxyguanosine, deoxythymidine, and deoxycytidine. Examples of well-known internucleoside linkages include phosphate diester groups, —OP(═O)(OH)O—; phosphate triester groups, —OP(═O)(OR′)O—; phosphite diester groups, —OP(OH)O—; phosphite triester groups, —OP(OR′)O—; and phosphonate groups, —OP(═O)(R)O—.

Oligonucleotides may conveniently be considered to be “nucleotide” polymers, that is, polymers comprised of nucleotide monomer units. The oligonucleotide may be linear, branched, or cyclic, but is typically linear. The term “nucleotide” conventionally refers to a phosphate ester of a nucleoside; that is, a chemical moiety comprising a phosphate group (i.e., —OP(═O)(OR)₂, where each R may independently be —H, a cation, or an organic group), a sugar moiety, and a nucleic acid base. Examples of well-known nucleotides formed from ribose and adenine, guanine, uracil, and cytosine are, respectively, the “5′(or 3′)-ribonucleotides”: adenosine 5′(or 3′)-monophosphate, guanosine 5′(or 3′)-monophosphate, uridine 5′(or 3′)-monophosphate, and cytidine 5′(or 3′)-monophosphate. Examples of well-known nucleotides formed from deoxyribose and adenine, guanine, thymine, and cytosine are respectively, the “5′(or 3′)-2′-deoxyribonucleotides”: deoxyadenosine 5′(or 3′)-monophosphate, deoxyguanosine 5′(or 3′)-monophosphate, deoxythymidine 5′(or 3′)-monophosphate, and deoxycytidine 5′(or 3′)-monophosphate.

Deoxyribonucleic acid (DNA) and ribonucleic acid (RNA) are important examples of oligonucleotides. In these oligonucleotides, all internucleoside bridges are phosphate diester linkages (—OP(═O)(OH)O—) and are uniformly in a 5′-3′ orientation; DNA is a polymer of deoxyribonucleotides involving primarily the nucleic acid bases adenine, guanine, cytosine, and thymine, whereas RNA is a polymer of ribonucleotides involving primarily the nucleic acid bases adenine, guanine, cytosine, and uracil. As a matter of convention, simple ribo- and deoxyribonucleotides and their polymers are represented by their bases (i.e., A, G, C, and U; or dA, dG, dC, and T (or dT), respectively), listed from the 5′-end of the oligonucleotide to the 3′-end of the oligonucleotide, wherein “d” denotes a deoxyribonucleotide. (Note that thymidine is often referred to “deoxythymidine” and abbreviated as “dT”.) For example, dGT is the dinucleotide of formed from deoxyguanosine and deoxythymidine, wherein the deoxyguanosine 5′-position is 5′-OH; the 3′-position of deoxyguanosine is linked to the 5′-position of the thymidine via a phosphate diester group; and the 3′-position of the thymidine is 3′-OH.

Oligosynthesis Using the Universal Solid Support

The universal solid support may he used in any method of solid-phase oligosynthesis capable of utilizing an protected or unprotected oligosynthesis group, Y, for example, —OH, —NH², —SH, or a protected form thereof. For example, the universal solid support may be used in solid-phase oligonucleotide synthesis.

Typically, the first step of any oligonucleotide synthesis method is an acid treatment of a functionalized solid support, to deprotect a functional group (e.g., an oligosynthesis group) for reaction with the first monomer reagent. Thus, if the oligosynthesis group, Y, is in a protected form, is it preferably deprotected by the same acid treatment used in oligonucleotide synthesis cycle.

Solid-phase oligonucleotide syntheses initially employed the use of phosphate triesters (the “triester method”) or phosphites (the “phosphite method”). With the discovery of relatively stable mononucleoside phosphoramidite coupling units (see, for example, Beaucage et al., 1981), solid-phase oligonucleotide synthesis became practical and common. Typical solid-phase oligonucleotide synthesis involves reiteratively performing four steps: deprotection, coupling, capping, and oxidation.

Standard methods involve stepwise synthesis of the oligonucleotide in the 5′-direction (i.e., 365). In the first step (“deprotection”), the growing oligonucleotide, which is attached at the 3′-end via a 3′-O group to a solid support, is 5′-deprotected to provide a reactive group (i.e., a 5′-OH group). For example, the 5′-OH group is often protected by reaction with 4,4′-dimethoxytrityl chloride (DMT-Cl) in pyridine, to yield a 5′-O-DMT group, which is stable under basic conditions, but which is easily deprotected under acid conditions, for example, in the presence of dichloroacetic acid (DCA) or trichloroacetic acid (TCA).

In the second step (“coupling”), the 5′-deprotected supported oligonucleotide is reacted with the desired nucleotide monomer, which itself has first been converted to a 5′-protected, 3′-phosphoramidite. For example, the 5′-OH group may be protected in the form of a 5′-ODMT group and the 3′-OH group may converted to a 3′-phosphoramidite, such as —OP(OR′)NR₂, where R is, for example, an isopropyl group, —CH(CH₃)₂, an ethyl group, —CH₂CH₃, or a methyl group, —CH₃, and R′ is, for example, —H (yielding a phosphoramidite diester), or —CH₃, —CH₂CH₃, the ∃-cyanoethyl group, —CH₂CH₂CN, or —C₆H₄Cl (yielding a phosphoramidite triester). The 3′-phosphoramidite group of the monomer reacts with the deprotected 5′-OH group of the growing oligonucleotide to yield the phosphite linkage 5′-OP(OR′)O-3′. See, for example, Caruthers et al., 1995.

Not all of the growing oligonucleotides will couple with the provided monomer; those which have not “grown” would yield incomplete oligonucleotides and therefore must be withdrawn from further synthesis. This is achieved by the third step (“capping”), in which all remaining —OH groups (i.e., unreacted 5′-OH groups) are capped, for example, in the form of acetates (5′-OC(O)CH₃,) by reaction with acetic anhydride (CH₃C(═O)—O—C(═O)CH₃). Note that, in some cases, this deprotection step follows, rather than precedes, the oxidation step discussed below.

In the fourth step (“oxidation”), the newly formed phosphite group (i.e., 5′-OP(OR′)O-3′) of the growing oligonucleotide is converted to a phosphate group (i.e., 5′-OP(═O)(OR′)O-3′), for example, by reaction with aqueous iodine and pyridine.

The four-step process may then be reiterated, since the oligonucleotide obtained after oxidation remains 5′-protected (e.g., 5′-ODMT) and is ready for use in the first deprotection step described above.

As example illustrating the first cycle of a phosphoramidite oligonucleotide synthesis method, starting with one preferred universal solid support of the present invention, is shown in FIG. 1.

Although the phosphoramidite method has been discussed in detail above, the universal solid support described herein may also be used in other methods of solid-phase oligonucleotide synthesis. Examples of such methods include, but are not limited to, H-phosphonate methods (also known as phosphite methods; see, for example, Froehler et al., 1990; Froehler et al., 1986); and phosphate triester methods (see, for example, Stec et al., 1985; Gallo et al., 1986; Gait, 1984).

The universal solid support described herein may also be used in other methods of solid-phase oligosynthesis to yield oligonucleotide derivatives and analogs. Examples of such products include, but are not limited to: DNA and RNA with various modifications at the 3′- and/or 5′-terminus; oligonucleoside methylphosphonates (Agarwal et al., 1987); phosphorothioate (Beaucage et al., 1990); phosphorodithioate (Bjergarde et al., 1991); DNA/RNA copolymers; phosphodiester/phosphorothioate copolymers; cyclic oligonucleotides (Wang et al., 1994); 2′-O-methyl RNA (Sproat et al, 1989); products containing peptide nucleic acids (Nielson et al, 1994); and, morpholine-type backbone modified nucleic acids (Stirchak et al, 1989; Summerton et al., 1991).

Cleavage from Solid Support

Following oligosynthesis, for example, when the desired oligonucleotide has been obtained, it may be cleaved from the solid support by treatment with a cleavage reagent in a cleavage step. The cleavage step is performed for a cleavage time, at a cleavage temperature.

The cleavage step achieves one or more, and preferably all, of the following results, in a single fast step:

(a) cleavage of the oligomer from the solid support, more particularly, cleavage of the bond between the bond between the linker group and the cyclic group.

(b) cleavage of the cyclic group from the oligomer, more particularly, cleavage of the bond between the phosphate and the oligomer. For example, a ∃-elimination reaction may yield a cyclic group with a cyclic phosphate group and the oligomer with a free —OH group.

(c) conversion of phosphate triesters (—OP(═O)(OR′)O— where R′ is not H) to the phosphate diesters (—OP(═O)₂O—), if present. Phosphate triesters may be present, for example, if a phosphoramidite synthesis method was used.

(d) deprotection of base-labile protected amino groups of nucleotide bases, if present. For example, if the primary amines of adenine, guanine, and cytosine had been protected as amides during oligosynthesis, they may be deprotected to yield free amino groups.

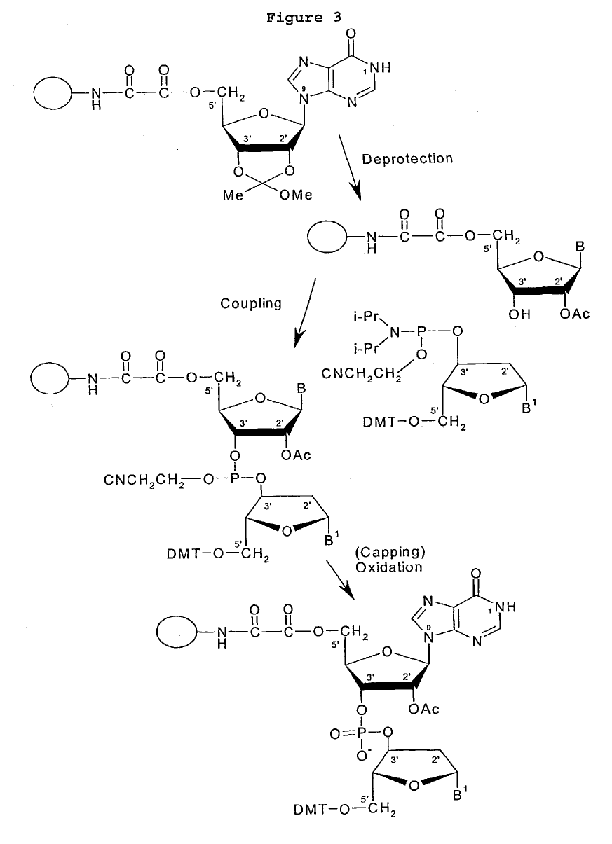

Two examples of the cleavage step are shown in FIGS. 2 and 3. In FIG. 2, the oligomer is a oligonucleotide grown in a 3′65′ direction, and attached to the solid support via a 5′-O group. The oligonucleotide cleavage product has a 5′-OH group. In FIG. 3, the oligomer is a oligonucleotide grown in a 5′63′ direction, and attached to the solid support via a 3′-O group. The oligonucleotide cleavage product has a 3′-OH group.

The cleavage is achieved by contacting the universal solid support bearing the oligonucleotides with a suitable cleavage reagent. In one embodiment, the cleavage reagent comprises one or more of the following: (a) a tertiary amine (NR₃), (b) a secondary amine (NHR₂), (c) a primary amine (RNH₂), and (d) ammonium hydroxide (NH₄OH).

Examples of suitable tertiary amines include, but are not limited to, triethylamine, diisopropylethylamine, N-methylpiperidine, and N-methylpyrrolidine. Examples of suitable secondary amines include, but are not limited to, dimethylamine, diethylamine, diisopropylamine, and piperidine. Examples of suitable primary amines include, but are not limited to, methylamine, ethylamine, and n-propylamine.

The relative amounts of (a) through (d) in the cleavage reagent may vary considerably, for example, from 1% to 99%, on a molar basis, if present. One of skill in the art is readily able to determine acceptable or optimum amounts.

In one preferred embodiment, the cleavage reagent comprises a mixture of (i) a tertiary amine (NR₃) or a secondary amine (NHR₂), and (ii) a primary amine (RNH₂), or ammonium hydroxide (NH₄OH).

In one preferred embodiment, the cleaving reagent comprises methylamine. In one preferred embodiment, the cleaving reagent comprises methylamine and one or more other components.

Suitable relative amounts of reagents and reaction conditions (e.g., temperature, time, and the like) may readily be determined by the skilled artisan.

Preferably, the cleavage temperature is above room temperature. In one embodiment, the cleavage temperature is 30 EC or greater (e.g., 30-100 EC). In one embodiment, the cleavage temperature is 50 EC or greater (e.g., 50-100 EC). In one embodiment, the cleavage temperature is 60 EC or greater (e.g., 60-100 EC). In one embodiment, the cleavage temperature is 70 EC or greater (e.g., 70-100 EC). In one embodiment, the cleavage temperature is 80 EC or greater (e.g., 80-100 EC).

The cleavage time will vary according the cleavage reagent and cleavage temperature chosen. Typically, the cleavage time is from 15 min to 24 hours. At a temperature of about 75 EC, the cleavage time is about 1 hr.

As discussed above, in one preferred embodiment, the linker group, R^{L}, is a covalent bond, and the linker reagent is an oxalyl dihalide. In this embodiment, the resulting oxalyl linkage is particularly advantageous in that (a) it has a high reactivity imparted by an ester group adjacent to a carbonyl group, and as a consequence of stereoelectronic effects related to this positioning, it is highly susceptible to cleavage by nucleophiles such as hydroxide, ammonia, and primary and secondary amines; and (b) it is stable to tertiary amines (for example, pyridine, lutidine) which are used in conventional DNA and RNA synthesis.

Loading

The universal solid supports of the present invention may be prepared with a high loading. In one embodiment, the solid support has a loading of more than 30 μmole/g (e.g., 30-100 μmole/g). In one embodiment, the solid support has a loading of more than 40 μmole/g (e.g., 40-100 μmole/g). In one embodiment, the solid support has a loading of more than 50 μmole/g (e.g., 50-100 μmole/g). In one embodiment, the solid support has a loading of 40-70 μmole/g.

### EXAMPLE

The following example is provided solely to illustrate the present invention and is not intended to limit the scope of the invention, as described herein.

A universal solid support was prepared as described below, and as illustrated in the chemical scheme in FIG. 2.

Oven-dried (100 EC, 30 min) aminopropyl-CPG (100 g, 250 μmole/g, Cruachem®) was suspended in 500 mL (727.7 g) of oxalyl chloride (ClC(═O)—C(═O)Cl, Aldrich®) in a 2 litre flask using a rotavapor system (R-114, Büchi®), at high speed (240 rev/min) at room temperature under reduced pressure (approx. 500 mbar). After 10 min, the mixture was heated to 100 EC over 120 min. Excess oxalyl chloride was evaporated after about 2.5 hr, to give a dry activated CPG (i.e., CPG with pendant linker groups).

A fresh solution of (a) 2′-3′-O-O-methoxyethylidene inosine in (b) dry pyridine and (c) N-methylimidazole (400 mL, 6.7 g of (a); 360 mL of (b), and 40 mL of (c)) was prepared immediately before use.

The above solution was added to the activated CPG in the rotavapor flask at high speed (240 rev/min) and at room temperature. The slurry was mixed for 20 min under reduced pressure (approx. 500 mbar).

40 mL of acetic anhydride (10% of the volume of inosine derivative) was added to the mixture, and the mixture allowed to stand for 5 min a high speed (240 rev/min) under reduced pressure (approx. 500 mbar).

The resulting beads were filtered on a glass-fritted funnel and washed several times with pyridine (2×300 mL) and acetonitrile (2×300 mL). The beads were dried (room temperature) in vacuum and stored under vacuum at 4 EC.

The 100 g of universal CPG thus obtained. The loading of this CPG was controlled and showed that it was 60 μmole/g.

### References

A number of patents and publications are cited above in order to more fully describe and disclose the invention and the state of the art to which the invention pertains. Full citations for these references are provided below. Each of these references is incorporated herein by reference in its entirety into the present disclosure.

Agarwal et al., 1987, Tetrahedron Letters, Vol. 28, p. 3539.

Arnold, 1996, “Oligonucleotide Polymeric Support System,” U.S. Pat. No. 5,539,097 issued 23 Jul. 1996.

Arnold, 1994, “Oligonucleotide Polymeric Support System with an Oxidation Cleavable Link,” U.S. Pat. No. 5,362,866 issued 08 Nov. 1994.

Beaucage et al., 1990, J. Am. Chem. Soc., Vol. 112, p.1253.

Beaucage et al., 1981, Tetrahedron Letters, Vol. 22, pp. 1859-1862.

Beaucage et al., 1992, “Title,” Tetrahedron, Vol. 48, pp. 2223-2311.

Bjergarde et al., 1991, Nucleic Acids Research, Vol. 18, pp. 5843-5850.

Carruthers, 1995, U.S. Pat. No. 4,415,732, issued Nov. 15, 1995.

DeBear et al., 1987, “A Universal Bead Support for Oligonucleotide Synthesis,” Nucleosides & Nucleotides, Vol. 6, No. 5, pp. 831-830.

Eritja et al., 1991, Tetrahedron Letters, Vol. 32, pp. 1511-1514.

Froehler et al., 1990, U.S. Pat. No. 4,959,463 issued 25 Sep. 1990.

Froehler et al., 1986, Nucleic Acids Research, Vol. 14, pp. 5399-5407.

Gait, M. J., 1984, Oligonucleotide Synthesis: A practical Approach, IRL Press.

Gallo et al., 1986, Nucleic Acids Research, Vol. 14, pp. 7405-7420.

Koester et al. et al., 1990, U.S. Pat. No. 4,923,901 issued 08 May 1990.

Letsinger et al., 1992, “Labile Anchors for Solid Phase Polynucleotide Synthesis,” U.S. Pat. No. 5,112,962 issued 12 May 1992.

Lyttle et al., 1996, “A new Universal Linker for Solid Phase DNA Synthesis,” Nucleic Acids Research, Vol. 24, No. 14, pp. 2793-2798.

McLean et al., 1997, “Compounds,” U.S. Pat. No. 5,681,945 granted 28 Oct. 1997.

Nelson et al., 1989, “Bifunctional Oligonucleotide Probes Synthesized Using a Novel CPG Support are able to Detect Single Base Pair Mutations,” Nucleic Acids Research, Vol. 17, No. 18, pp. 7187-7193.

Nielson et al, 1994, Bioconjugate Chem, Vol. 5, p. 3.

Pon et al., 1997, Tetrahedron Letters, Vol. 38, pp. 3327-3330.

Reddy et al., 1997, “Universal Solid Supports and Methods for Their Use,” International Patent Publication number WO 97/40458, published 30 Oct. 1997.

Scheuer-Larsen et al., 1997, “Introduction of a Universal Solid Support for Oligonucleotide Synthesis,” Nucleosides and Nucleotides, Vol. 16, Nos. 1&2, pp. 67-80.

Schwartz et al., 1995, “A Universal Adapter for Chemical Synthesis of DNA or RNA on any Single Type of Solid Support,” Tetrahedron Letters, Vol. 36, No. 1, pp. 27-30.

Sproat et al, 1989, Nucleic Acids Research, Vol. 18, pp. 3373-3386.

Stec et al., 1985, Tetrahedron Letters, Vol. 26, p. 2191.

Stirchak et al, 1989, Nucleic Acids Research, Vol. 17, pp. 6129-6141.

Summerton et al., 1991, U.S. Pat. No. 5,034,506 issued 23 Jul. 1991.

Tracy et al., 1997, Bioconjugate Chemistry, Vol. 8, pp. 99-102.

Vu et al., 1995, “Use of Phthaloyl Protecting Group for the Automated Synthesis of 3′-[(Hydroxypropyl)amino] and 3′-[(Hydroxpropyl)triglycyl]Oligonucleotide Conjugates,” Bioconjugate Chem., Vol. 6, pp. 599-607.

Wang et al., 1994, Nucleic Acids Research, Vol. 22, pp. 2326-2333.

Webb et al., 1987, “Support for Solid-Phase Oligonucleotide Synthesis,” U.S. Pat. No. 4,659,774 issued 21 Apr. 1987.

1. A method for the preparation of a solid support suitable for use in solid-phase oligosynthesis, which method comprises the steps of:
(a) reacting a pendant functional group of a solid support with a linker reagent, thereby forming a pendant linker group;
(b) removing at least a portion of excess unreacted linker reagent by evaporation; and,
(c) reacting said pendant linker group with a cyclic reagent, thereby forming a pendant cyclic group.

2. A method for the preparation of a solid support suitable for use in solid-phase oligonucleotide synthesis, which method comprises the steps of:
(a) reacting a pendant functional group of a solid support with a linker reagent, thereby forming a pendant linker group;
(b) removing at least a portion of excess unreacted linker reagent by evaporation;
(c) reacting said pendant linker group with a cyclic reagent, thereby forming a pendant cyclic group; and,
(d) removing at least a portion of excess unreacted cyclic reagent and/or by-products thereof.

3. A method according to claim 1 or 2, further comprising, after said step of reacting a pendant functional group of a solid support with a-linker reagent, a step of:
(i) reacting at least a portion of any remaining pendant functional groups of said solid support with a capping reagent, thereby forming pendant capped functional groups.

4. A method according to any one of claims 1 to 3, further comprising, after said step of removing at least a portion of excess unreacted linker reagent by evaporation, a step of:
(i) reacting at least a portion of any remaining pendant functional groups of said solid support with a capping reagent, thereby forming pendant capped functional groups.

5. A method according to any one of claims 1 to 4, further comprising, after said step of reacting said pendant linker group with a cyclic reagent, a step of:
(i) reacting at least a portion of any remaining pendant linker groups of said solid support with a capping reagent, thereby forming pendant capped linker groups.

6. A method according to any one of claims 3, 4, and 5, wherein said capping reagent is acetic anhydride.

7. A method according to any one of claims 1 to 6, wherein said step of removing at least a portion of excess unreacted linker reagent by evaporation is achieved with the application of reduced pressure and/or increased temperature.

8. A method according to any one of claims 1 to 7, wherein said step of removing at least a portion of excess unreacted linker reagent by evaporation involves removing a substantial portion of excess unreacted linker reagent.

9. A method according to any one of claims 1 to 8, wherein said step of removing at least a portion of excess unreacted linker reagent is achieved solely by evaporation.

10. A method according to any one of claims 1 to 9, wherein one or more steps of said method are performed using an evaporation apparatus.

11. A method according to any one of claims 1 to 9, wherein said method is performed using a rotavapor apparatus or a distillation apparatus.

12. A method according to any one of claims 1 to 9, wherein said method is performed using a rotavapor apparatus.

13. A method according to any one of claims 1 to 12, wherein said linker reagent has the formula: 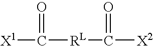
wherein
X¹ and X² are independently —Cl, —Br, or —I; and,
R^{L} denotes a covalent bond or a divalent group which is an organic group comprising from 1 to 10 carbon atoms and from 0 to 5 heteroatoms selected from N, O, and S; and wherein said linker reagent is a volatile liquid.

14. A method according to any one of claims 1 to 12, wherein said linker reagent has the formula: 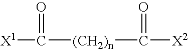
wherein X¹ and X² are independently —Cl, —Br, or —I, and n is an integer from 0 to 2.

15. A method according to claim 14, wherein said linker reagent is one of:

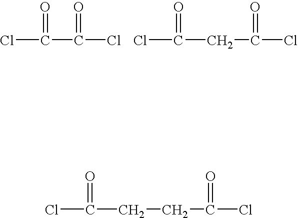

16. A method according to claim 14, wherein said linker reagent is:

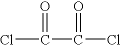

17. A method according to any one of claims 1 to 16, wherein said pendant functional group of said solid support is -J¹-H, wherein J¹ is —NH—, —O—, or —S—, denoted as follows:

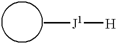

18. A method according to claim 17, wherein said pendant functional group of said solid support is —NH₂, denoted as follows:

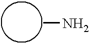

19. A method according to any one of claims 1 to 18, wherein said cyclic reagent has the formula: 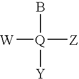
wherein:
Q denotes a cyclic group, which has a single non-aromatic ring, which ring has from 5 to 7 ring atoms, which ring atoms are:
(a) all carbon atoms; or,
(b) carbon atoms and one or two heteroatoms selected from oxygen, nitrogen, and sulfur;
W denotes a reactive conjugating group and is —OH, —NH₂, or —SH;
B denotes a base moiety, which is a purine or pyrimidine or a derivative or analog thereof;
Y denotes an oligosynthesis group, which is —OH, —NH₂, or —SH, or a protected form thereof; and,
Z, if present, denotes an auxiliary group, which is —OH, —NH₂, or —SH, or a protected form thereof;
wherein said W, B, Y, and Z, if present, are each separately attached to a carbon ring atom of said Q, either directly, via a covalent bond, or indirectly, via an intermediate covalent linkage selected from —CH₂— and —CH₂CH₂—.

20. A method according to claim 19, wherein Q is selected from:

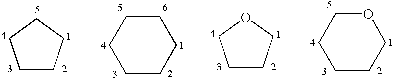

21. A method according to claim 20, the moiety Y-Q-Z of the cyclic reagent has a structure selected from:

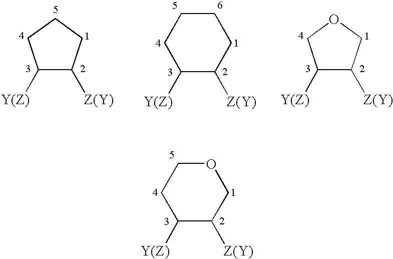

22. A method according to claim 21, the moiety Y-Q-Z of the cyclic reagent has a structure selected from:

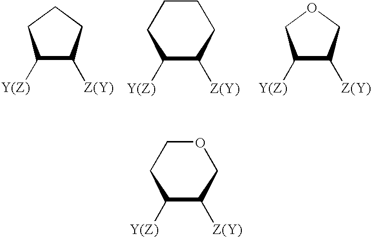

23. A method according to claim 22, wherein the cyclic reagent is selected from:

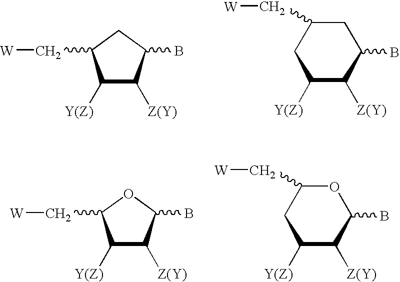

24. A method according to claim 22, wherein the cyclic reagent is selected from:

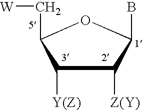

25. A method according to any one of claims 1 to 24, wherein B is selected from the following, or a protected form thereof:

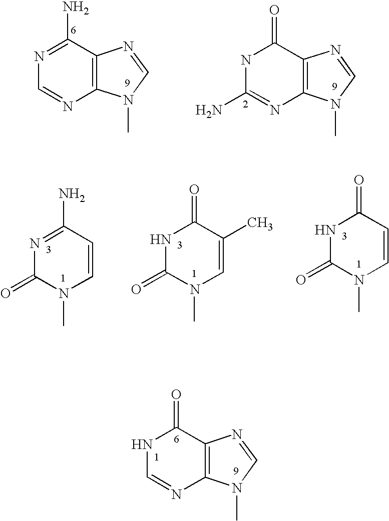

26. A method according to claim 25, wherein B is the following, or a protected form thereof:

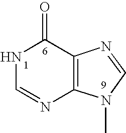

27. A method according to any one of claims 1 to 26, wherein W is —OH.

28. A method according to any one of claims 1 to 27, wherein Y is —OH or a protected form thereof.

29. A method according to any one of claims 1 to 28, wherein Z, if present, is —OH or a protected form thereof.

30. A method according to any one of claims 1 to 29, wherein Z, if present, is —OH protected in the form of —OR, wherein R is an acyl group, a C₁₋₇alkyl group, or a silyl group.

31. A method according to any one of claims 1 to 30, wherein both Z and Y, in deprotected form, are —OH, and, in protected form, together form part of a cyclic structure with an O—O-methoxyethylidene group of the structure:

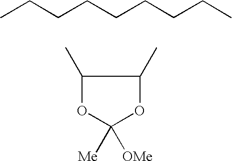

32. A method according to any one of claims 1 to 29, wherein said cyclic reagent is: 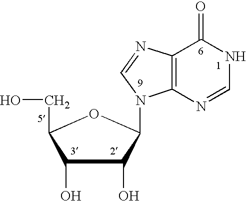
or a protected form thereof.

33. A method according to claim 32, wherein said cyclic reagent is:

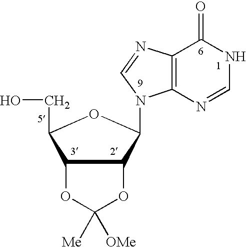

34. A method according to any one of claims 1 to 33, wherein said step of reacting said pendant linker group with a cyclic reagent is performed in the present of an added base.

35. A method according to claim 34, wherein said added base is dimethylaminopyridine (DMAP) or N-methylimidazole (NMI).

36. A solid support of the formula: 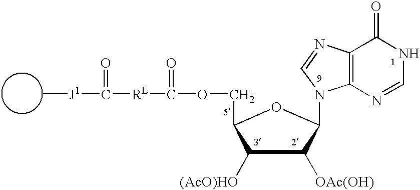
wherein:
J¹ is —NH—;
R^{L} is —(CH₂)ₙ—; and,
n is an integer from 0 to 10;
or a protected form thereof.

37. A solid support of the formula: 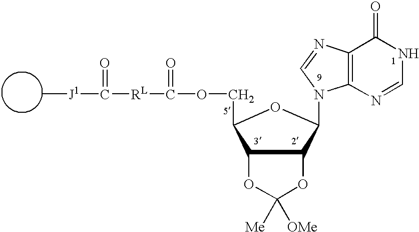
wherein:
J¹ is —NH—;
R^{L} is —(CH₂)ₙ—; and,
n is an integer from 0 to 10.

38. A solid support of the formula: 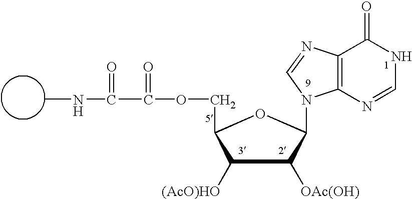
or a protected form thereof.

39. A solid support of the formula:

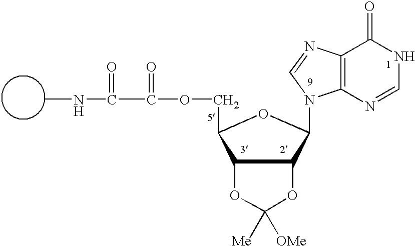

40. A solid support suitable for use in solid-phase oligonucleotide synthesis prepared by a method as described in any one of claims 1 to 35.

41. A method of oligonucleotide synthesis, which method employs a method for the preparation of a universal solid support as described in any one of claims 1 to 35.

42. A method of oligonucleotide synthesis, which method employs a universal solid support as described in any one of claims 36 to 39.

43. An oligonucleotide which has been prepared using a method of oligonucleotide synthesis, which method employs a method for the preparation of a universal solid support as described in any one of claims 1 to 35.

44. An oligonucleotide which has been prepared using a universal solid support as described in any one of claims 36 to 39.
